SEQUENCE LISTING (110 sequences)

SEQ ID NO: 1 - RNA, 1542 nt, Escherichia coli
aaauugaagaguuugaucauggcucagauugaacgcuggcggcaggccuaacacaugcaagucgaacgguaacaggaaacagcuugcugcuucgcugacgaguggcggacgggugaguaaugucugggaaacugccugauggagggggauaacuacuggaaacgguagcuaauaccgcauaacgucgcaagaccaaagagggggaccuuagggccucuugccaucggaugugcccagaugggauuagcuaguaggugggguaacggcucaccuaggcgacgaucccuagcuggucugagaggaugaccagccacacuggaacugagacacgguccagacuccuacgggaggcagcaguggggaauauugcacaaugggcgcaagccugaugcagccaugccgcguguaugaagaaggccuucggguuguaaaguacuuucagcggggaggaagggaguaaaguuaauaccuuugcucauugacguuacccgcagaagaagcaccggcuaacuccgugccagcagccgcgguaauacggagggugcaagcguuaaucggaauuacugggcguaaagcgcacgcaggcgguuuguuaagucagaugugaaauccccgggcucaaccugggaacugcaucugauacuggcaagcuugagucucguagagggggguagaauuccagguguagcggugaaaugcguagagaucuggaggaauaccgguggcgaaggcggcccccuggacgaagacugacgcucaggugcgaaagcguggggagcaaacaggauuagauacccugguaguccacgccguaaacgaugucgacuuggagguugugcccuugaggcguggcuuccggagcuaacgcguuaagucgaccgccuggggaguacggccgcaagguuaaaacucaaaugaauugacgggggcccgcacaagcgguggagcaugugguuuaauucgaugcaacgcgaagaaccuuaccuggucuugacauccacggaaguuuucagagaugagaaugugccuucgggaaccgugagacaggugcugcauggcugucgucagcucguguugugaaauguuggguuaagucccgcaacgagcgcaacccuuauccuuuguugccagcgguccggccgggaacucaaaggagacugccagugauaaacuggaggaagguggggaugacgucaagucaucauggcccuuacgaccagggcuacacacgugcuacaauggcgcauacaaagagaagcgaccucgcgagagcaagcggaccucauaaagugcgucguaguccggauuggagucugcaacucgacuccaugaagucggaaucgcuaguaaucguggaucagaaugccacggugaauacguucccgggccuuguacacaccgcccgucacaccaugggaguggguugcaaaagaaguagguagcuuaaccuucgggagggcgcuuaccacuuugugauucaugacuggggugaagucguaacaagguaaccguaggggaaccugcgguuggaucaccuccuua

SEQ ID NO: 2 - DNA, 32 bp, Artificial Sequence
  Synthesized
ctacctacttcttttgattcaccgtggcattc

SEQ ID NO: 3 - DNA, 32 bp, Artificial Sequence
  Synthesized
ccctctacgagactcaggtattaactttactc

SEQ ID NO: 4 - DNA, 34 bp, Artificial Sequence
  Synthesized
aagcagcaagctgtttcggtccccctctttggtc

SEQ ID NO: 5 - DNA, 34 bp, Artificial Sequence
  Synthesized
gggcccccgtcaattcactgtctcacggttcccg

SEQ ID NO: 6 - DNA, 32 bp, Artificial Sequence
  Synthesized
gcggtcgacttaacgctttacggcgtggacta

SEQ ID NO: 7 - DNA, 32 bp, Artificial Sequence
  Synthesized
cacaaagtggtaagcgcgattccgacttcatg

SEQ ID NO: 8 - DNA, 34 bp, Artificial Sequence
  Synthesized
atcgaattaaaccacattctgaaaacttccgtgg

SEQ ID NO: 9 - DNA, 34 bp, Artificial Sequence
  Synthesized
gtgcaatattccccacttaacgtcaatgagcaaa

SEQ ID NO: 10 - DNA, 34 bp, Artificial Sequence
  Synthesized
aagggccatgatgacttgagtcgagttgcagact

SEQ ID NO: 11 - DNA, 34 bp, Artificial Sequence
  Synthesized
gcctgcgtgcgctttaccctccaagtcgacatcg

SEQ ID NO: 12 - DNA, 34 bp, Artificial Sequence
  Synthesized
gcggccgtactccccagtcacaacacgagctgac

SEQ ID NO: 13 - DNA, 17 bp, Artificial Sequence
  Synthesized
tcaccccagtcatgaat

SEQ ID NO: 14 - DNA, 34 bp, Artificial Sequence
  Synthesized
ttcccggccggaccgctacaaggcccgggaacgt

SEQ ID NO: 15 - DNA, 34 bp, Artificial Sequence
  Synthesized
gtctggaccgtgtctcatggcacggagttagccg

SEQ ID NO: 16 - DNA, 34 bp, Artificial Sequence
  Synthesized
tcatcctctcagaccagtaacgcttgcaccctcc

SEQ ID NO: 17 - DNA, 32 bp, Artificial Sequence
  Synthesized
gacgtcatccccacctgacagccatgcagcac

SEQ ID NO: 18 - DNA, 34 bp, Artificial Sequence
  Synthesized
agcccggggatttcacaccagggtatctaatcct

SEQ ID NO: 19 - DNA, 16 bp, Artificial Sequence
  Synthesized
ctgttaccgttcgact

SEQ ID NO: 20 - DNA, 32 bp, Artificial Sequence
  Synthesized
tttgagttttaaccttgtttgctccccacgct

SEQ ID NO: 21 - DNA, 32 bp, Artificial Sequence
  Synthesized
caggcttgcgcccattttgcgacgttatgcgg

SEQ ID NO: 22 - DNA, 32 bp, Artificial Sequence
  Synthesized
tgcagttcccaggttggtgcttcttctgcggg

SEQ ID NO: 23 - DNA, 34 bp, Artificial Sequence
  Synthesized
gttagctccggaagccaggttgcgctcgttgcgg

SEQ ID NO: 24 - DNA, 34 bp, Artificial Sequence
  Synthesized
acgcggcatggctgcatccttcctccccgctgaa

SEQ ID NO: 25 - DNA, 34 bp, Artificial Sequence
  Synthesized
cacctggaattctacccccgccttcgccaccggt

SEQ ID NO: 26 - DNA, 17 bp, Artificial Sequence
  Synthesized
ccctcccgaaggttaag

SEQ ID NO: 27 - DNA, 34 bp, Artificial Sequence
  Synthesized
agacattactcacccgttattagctaccgtttcc

SEQ ID NO: 28 - DNA, 34 bp, Artificial Sequence
  Synthesized
agcttgccagtatcagattcgcacctgagcgtca

SEQ ID NO: 29 - DNA, 32 bp, Artificial Sequence
  Synthesized
gctccaccgcttgtgcgtcttcgtccaggggg

SEQ ID NO: 30 - DNA, 34 bp, Artificial Sequence
  Synthesized
tgcatgtgttaggcctgcatctgggcacatccga

SEQ ID NO: 31 - DNA, 32 bp, Artificial Sequence
  Synthesized
gttccagtgtggctggacctactagctaatcc

SEQ ID NO: 32 - DNA, 34 bp, Artificial Sequence
  Synthesized
tgagccatgatcaaactaggtgagccgttacccc

SEQ ID NO: 33 - DNA, 32 bp, Artificial Sequence
  Synthesized
gtgtagccctggtcgtaaggcacattctcatc

SEQ ID NO: 34 - DNA, 32 bp, Artificial Sequence
  Synthesized
gctgcctcccgtaggatggcaagaggccctaa

SEQ ID NO: 35 - DNA, 32 bp, Artificial Sequence
  Synthesized
ggcagtctcctttgaggacttaacccaacatt

SEQ ID NO: 36 - DNA, 34 bp, Artificial Sequence
  Synthesized
gcgccattgtagcacgtcgcactttatgaggtcc

SEQ ID NO: 37 - DNA, 34 bp, Artificial Sequence
  Synthesized
tcctccagtttatcacttgatccacgattactag

SEQ ID NO: 38 - DNA, 17 bp, Artificial Sequence
  Synthesized
cgcaggttcccctacgg

SEQ ID NO: 39 - DNA, 16 bp, Artificial Sequence
  Synthesized
ccgccagcgttcaatc

SEQ ID NO: 40 - DNA, 32 bp, Artificial Sequence
  Synthesized
tctgacttaacaaaccgtattaccgcggctgc

SEQ ID NO: 41 - DNA, 32 bp, Artificial Sequence
  Synthesized
ttaccttgttacgactccaatccggactacga

SEQ ID NO: 42 - DNA, 16 bp, Artificial Sequence
  Synthesized
ccgccactcgtcagcg

SEQ ID NO: 43 - DNA, 32 bp, Artificial Sequence
  Synthesized
ttttttttcggtgtgtggcaacaatttttttt

SEQ ID NO: 44 - DNA, 33 bp, Artificial Sequence
  Synthesized
caacccactcccatggtgtgacgggtttttttt

SEQ ID NO: 45 - DNA, 48 bp, Artificial Sequence
  Synthesized
tttttttttcgcttctctttgtatatgtcaagaccaggtatttttttt

SEQ ID NO: 46 - DNA, 32 bp, Artificial Sequence
  Synthesized
ttttttttaggataagcgcctcaatttttttt

SEQ ID NO: 47 - DNA, 32 bp, Artificial Sequence
  Synthesized
ttttttttgggcacaagcccagtatttttttt

SEQ ID NO: 48 - DNA, 48 bp, Artificial Sequence
  Synthesized
ttttttttacgcatttcaccgctaagtactttacaacccgtttttttt

SEQ ID NO: 49 - DNA, 48 bp, Artificial Sequence
  Synthesized
ttttttttaaggccttcttcatacagtagttatccccctctttttttt

SEQ ID NO: 50 - DNA, 48 bp, Artificial Sequence
  Synthesized
ttttttttaggttcttcgcgttgcattcctccagatctcttttttttt

SEQ ID NO: 51 - DNA, 32 bp, Artificial Sequence
  Synthesized
ttttttttcgtcgcctcttcaatttttttttt

SEQ ID NO: 52 - DNA, 48 bp, Artificial Sequence
  Synthesized
ttttttttaaggaggtgatccaacgcttgctctcgcgaggtttttttt

SEQ ID NO: 53 - DNA, 32 bp, Artificial Sequence
  Synthesized
ttttttttattccgatctagggattttttttt

SEQ ID NO: 54 - DNA, 24 bp, Artificial Sequence
  Synthesized
ttttttttcatcaggcagtttccc

SEQ ID NO: 55 - DNA, 84 bp, Artificial Sequence
  Synthesized
ccgccactcgtcagcgaagcagcaagctgtttcggtccccctctttggtcgtgcaatattccccacttaacgtcaatgagcaaa

SEQ ID NO: 56 - DNA, 80 bp, Artificial Sequence
  Synthesized
ttaccttgttacgactccaatccggactacgagtgtagccctggtcgtaaggcacattctcatcgctccaccgcttgtgc

SEQ ID NO: 57 - DNA, 62 bp, Artificial Sequence
  Synthesized
ttcccggccggaccgctacaaggcccgggaacgtcaacccactcccatggtgtgacgggttt

SEQ ID NO: 58 - DNA, 85 bp, Artificial Sequence
  Synthesized
agcttgccagtatcagattcgcacctgagcgtcagggcccccgtcaattcactgtctcacggttcccgaagggccatgatgactt

SEQ ID NO: 59 - DNA, 64 bp, Artificial Sequence
  Synthesized
tctgacttaacaaaccgtattaccgcggctgcgttccagtgtggctggacctactagctaatcc

SEQ ID NO: 60 - DNA, 64 bp, Artificial Sequence
  Synthesized
ggcagtctcctttgaggacttaacccaacattgcggtcgacttaacgctttacggcgtggacta

SEQ ID NO: 61 - DNA, 82 bp, Artificial Sequence
  Synthesized
gctgcctcccgtaggatggcaagaggccctaactgttaccgttcgacttgcatgtgttaggcctgcatctgggcacatccga

SEQ ID NO: 62 - DNA, 80 bp, Artificial Sequence
  Synthesized
gtcttcgtccagggggccctctacgagactcaggtattaactttactccaggcttgcgcccattttgcgacgttatgcgg

SEQ ID NO: 63 - DNA, 68 bp, Artificial Sequence
  Synthesized
gcctgcgtgcgctttaccctccaagtcgacatcggttagctccggaagccaggttgcgctcgttgcgg

SEQ ID NO: 64 - DNA, 68 bp, Artificial Sequence
  Synthesized
tctgaaaacttccgtgggcgccattgtagcacgtcgcactttatgaggtcccgcaggttcccctacgg

SEQ ID NO: 65 - DNA, 84 bp, Artificial Sequence
  Synthesized
ccgccagcgttcaatctgagccatgatcaaactaggtgagccgttacccctcatcctctcagaccagtaacgcttgcaccctcc

SEQ ID NO: 66 - DNA, 70 bp, Artificial Sequence
  Synthesized
tttcatcaggcagtttcccagacattactcacccgttattagctaccgtttccacgcggcatggctgcat

SEQ ID NO: 67 - DNA, 80 bp, Artificial Sequence
  Synthesized
gacagccatgcagcactttgagttttaaccttgtttgctccccacgcttgcagttcccaggttggtgcttcttctgcggg

SEQ ID NO: 68 - DNA, 85 bp, Artificial Sequence
  Synthesized
gtctggaccgtgtctcatggcacggagttagccgagcccggggatttcacaccagggtatctaatcctgcggccgtactccccag

SEQ ID NO: 69 - DNA, 68 bp, Artificial Sequence
  Synthesized
ccttcctccccgctgaacacctggaattctacccccgccttcgccaccggtatcgaattaaaccacat

SEQ ID NO: 70 - DNA, 82 bp, Artificial Sequence
  Synthesized
gagtcgagttgcagacttcaccccagtcatgaatcacaaagtggtaagcgcgattccgacttcatggacgtcatccccacct

SEQ ID NO: 71 - DNA, 100 bp, Artificial Sequence
  Synthesized
tcacaacacgagctgactcctccagtttatcacttgatccacgattactagccctcccgaaggttaagctacctacttcttttgattcaccgtggcattc

SEQ ID NO: 72 - DNA, 32 bp, Artificial Sequence; identical to SEQ ID NO: 53
  Synthesized

SEQ ID NO: 73 - DNA, 32 bp, Artificial Sequence; identical to SEQ ID NO: 43
  Synthesized

SEQ ID NO: 74 - DNA, 48 bp, Artificial Sequence; identical to SEQ ID NO: 49
  Synthesized

SEQ ID NO: 75 - DNA, 48 bp, Artificial Sequence; identical to SEQ ID NO: 45
  Synthesized

SEQ ID NO: 76 - DNA, 32 bp, Artificial Sequence; identical to SEQ ID NO: 47
  Synthesized

SEQ ID NO: 77 - DNA, 48 bp, Artificial Sequence; identical to SEQ ID NO: 52
  Synthesized

SEQ ID NO: 78 - DNA, 48 bp, Artificial Sequence; identical to SEQ ID NO: 50
  Synthesized

SEQ ID NO: 79 - DNA, 32 bp, Artificial Sequence; identical to SEQ ID NO: 51
  Synthesized

SEQ ID NO: 80 - DNA, 32 bp, Artificial Sequence; identical to SEQ ID NO: 46
  Synthesized

SEQ ID NO: 81 - DNA, 48 bp, Artificial Sequence; identical to SEQ ID NO: 48
  Synthesized

SEQ ID NO: 82 - DNA, 211 bp, Artificial Sequence
  Synthesized
ccgccagcgttcaatctgagccatgatcaaactaggtgagccgttacccctcatcctctcagaccagtaacgcttgcaccctccgcctgcgtgcgctttaccctccaagtcgacatcggttagctccggaagccaggttgcgctcgttgcggttcccggccggaccgctacaaggcccgggaacgtcaacccactcccatggtgtgacggg

SEQ ID NO: 83 - DNA, 203 bp, Artificial Sequence
  Synthesized
ccgccactcgtcagcgaagcagcaagctgtttcggtccccctctttggtcgtgcaatattccccacttaacgtcaatgagcaaaagcttgccagtatcagattcgcacctgagcgtcagggcccccgtcaattcactgtctcacggttcccgaagggccatgatgacttgagtcgagttgcagacttcaccccagtcatgaat

SEQ ID NO: 84 - DNA, 203 bp, Artificial Sequence
  Synthesized
catcaggcagtttcccagacattactcacccgttattagctaccgtttccacgcggcatggctgcatccttcctccccgctgaacacctggaattctacccccgccttcgccaccggtatcgaattaaaccacattctgaaaacttccgtgggcgccattgtagcacgtcgcactttatgaggtcccgcaggttcccctacgg

SEQ ID NO: 85 - DNA, 203 bp, Artificial Sequence
  Synthesized
ctgttaccgttcgacttgcatgtgttaggcctgcatctgggcacatccgagtctggaccgtgtctcatggcacggagttagccgagcccggggatttcacaccagggtatctaatcctgcggccgtactccccagtcacaacacgagctgactcctccagtttatcacttgatccacgattactagccctcccgaaggttaag

SEQ ID NO: 86 - DNA, 309 bp, Artificial Sequence
  Synthesized
taatacgactcactataggggaattgtgagcggataacaattccgccagcgttcaatctgagccatgatcaaactaggtgagccgttacccctcatcctctcagaccagtaacgcttgcaccctccgcctgcgtgcgctttaccctccaagtcgacatcggttagctccggaagccaggttgcgctcgttgcggttcccggccggaccgctacaaggcccgggaacgtcaacccactcccatggtgtgacgggctagcataaccccttggggcctctaaacgggtcttgaggggttttttgtttttttt

SEQ ID NO: 87 - DNA, 301 bp, Artificial Sequence
  Synthesized
taatacgactcactataggggaattgtgagcggataacaattccgccactcgtcagcgaagcagcaagctgtttcggtccccctctttggtcgtgcaatattccccacttaacgtcaatgagcaaaagcttgccagtatcagattcgcacctgagcgtcagggcccccgtcaattcactgtctcacggttcccgaagggccatgatgacttgagtcgagttgcagacttcaccccagtcatgaatctagcataaccccttggggcctctaaacgggtcttgaggggttttttgtttttttt

SEQ ID NO: 88 - DNA, 301 bp, Artificial Sequence
  Synthesized
taatacgactcactataggggaattgtgagcggataacaattcatcaggcagtttcccagacattactcacccgttattagctaccgtttccacgcggcatggctgcatccttcctccccgctgaacacctggaattctacccccgccttcgccaccggtatcgaattaaaccacattctgaaaacttccgtgggcgccattgtagcacgtcgcactttatgaggtcccgcaggttcccctacggctagcataaccccttggggcctctaaacgggtcttgaggggttttttgtttttttt

SEQ ID NO: 89 - DNA, 293 bp, Artificial Sequence
  Synthesized
taatacgactcactataggggaattgtgagcggataacaattctgttaccgttcgacttgcatgtgttaggcctgcatctgggcacatccgagtctggaccgtgtctcatggcacggagttagccgagcccggggatttcacaccagggtatctaatcctgcggccgtactccccagtcacaacacgagctgactcctccagtttatcacttgatccacgattactagccctcccgaaggttaagctagcataaccccttggggcctctaaacgggtcttgaggggttttttg

SEQ ID NO: 90 - DNA, 150 bp, Artificial Sequence
  Synthesized
accttccttttcatttcccttcctttccccctttttttcctctcctttctccctttatttctccttacccttcctacttctctctttccctcccctcctctactaccccatctttactcccctttcctatccctccaactcacactaaac

SEQ ID NO: 91 - DNA, 1690 bp, Artificial Sequence
  Synthesized
cgtacggaattcgctagctaatacgactcactataggggaattgtgagcggataacaattccgccagcgttcaatctgagccatgatcaaactaggtgagccgttacccctcatcctctcagaccagtaacgcttgcaccctccgcctgcgtgcgctttaccctccaagtcgacatcggttagctccggaagccaggttgcgctcgttgcggttcccggccggaccgctacaaggcccgggaacgtcaacccactcccatggtgtgacgggctagcataaccccttggggcctctaaacgggtcttgaggggttttttgttttttttaccttccttttcatttcccttcctttccccctttttttcctctcctttctccctttatttctccttacccttcctacttctctctttccctcccctcctctactaccccatctttactcccctttcctatccctccaactcacactaaactaatacgactcactataggggaattgtgagcggataacaattccgccactcgtcagcgaagcagcaagctgtttcggtccccctctttggtcgtgcaatattccccacttaacgtcaatgagcaaaagcttgccagtatcagattcgcacctgagcgtcagggcccccgtcaattcactgtctcacggttcccgaagggccatgatgacttgagtcgagttgcagacttcaccccagtcatgaatctagcataaccccttggggcctctaaacgggtcttgaggggttttttgtttttttttcttcactttaactcacactaccccacatccccctcctactcaaaaactcccctcccacaacccaaacccaccacaccaacaacaaccaaaactcacccaccccccctcactaacccaaaaaaaaaacaccaccaaccaaccataactactaatacgactcactataggggaattgtgagcggataacaattcatcaggcagtttcccagacattactcacccgttattagctaccgtttccacgcggcatggctgcatccttcctccccgctgaacacctggaattctacccccgccttcgccaccggtatcgaattaaaccacattctgaaaacttccgtgggcgccattgtagcacgtcgcactttatgaggtcccgcaggttcccctacggctagcataaccccttggggcctctaaacgggtcttgaggggttttttgtttttttttcaattacctctctctcatctcacctcctacaccacaccctcccaactaaccacacaccaacacactcaaacttctctctatccctcccaatcaccctactccgcgccttccccttcccttcagtttcccctttttctctctttcttcactaatacgactcactataggggaattgtgagcggataacaattctgttaccgttcgacttgcatgtgttaggcctgcatctgggcacatccgagtctggaccgtgtctcatggcacggagttagccgagcccggggatttcacaccagggtatctaatcctgcggccgtactccccagtcacaacacgagctgactcctccagtttatcacttgatccacgattactagccctcccgaaggttaagctagcataaccccttggggcctctaaacgggtcttgaggggttttttgggatccgagctccacgtg

SEQ ID NO: 92 - DNA, 288 bp, Artificial Sequence
  Synthesized
taatacgactcactataggggccgccagcgttcaatctgagccatgatcaaactaggtgagccgttacccctcatcctctcagaccagtaacgcttgcaccctccgcctgcgtgcgctttaccctccaagtcgacatcggttagctccggaagccaggttgcgctcgttgcggttcccggccggaccgctacaaggcccgggaacgtcaacccactcccatggtgtgacgggctagcataaccccttggggcctctaaacgggtcttgaggggttttttgtttttttt

SEQ ID NO: 93 - DNA, 280 bp, Artificial Sequence
  Synthesized
taatacgactcactataggggccgccactcgtcagcgaagcagcaagctgtttcggtccccctctttggtcgtgcaatattccccacttaacgtcaatgagcaaaagcttgccagtatcagattcgcacctgagcgtcagggcccccgtcaattcactgtctcacggttcccgaagggccatgatgacttgagtcgagttgcagacttcaccccagtcatgaatctagcataaccccttggggcctctaaacgggtcttgaggggttttttgtttttttt

SEQ ID NO: 94 - DNA, 280 bp, Artificial Sequence
  Synthesized
taatacgactcactataggggcatcaggcagtttcccagacattactcacccgttattagctaccgtttccacgcggcatggctgcatccttcctccccgctgaacacctggaattctacccccgccttcgccaccggtatcgaattaaaccacattctgaaaacttccgtgggcgccattgtagcacgtcgcactttatgaggtcccgcaggttcccctacggctagcataaccccttggggcctctaaacgggtcttgaggggttttttgtttttttt

SEQ ID NO: 95 - DNA, 272 bp, Artificial Sequence
  Synthesized
taatacgactcactataggggctgttaccgttcgacttgcatgtgttaggcctgcatctgggcacatccgagtctggaccgtgtctcatggcacggagttagccgagcccggggatttcacaccagggtatctaatcctgcggccgtactccccagtcacaacacgagctgactcctccagtttatcacttgatccacgattactagccctcccgaaggttaagctagcataaccccttggggcctctaaacgggtcttgaggggttttttg

SEQ ID NO: 96 - RNA, 211 nt, Artificial Sequence
  Synthesized
ccgccagcguucaaucugagccaugaucaaacuaggugagccguuaccccucauccucucagaccaguaacgcuugcacccuccgccugcgugcgcuuuacccuccaagucgacaucgguuagcuccggaagccagguugcgcucguugcgguucccggccggaccgcuacaaggcccgggaacgucaacccacucccauggugugacggg

SEQ ID NO: 97 - RNA, 203 nt, Artificial Sequence
  Synthesized
ccgccacucgucagcgaagcagcaagcuguuucggucccccucuuuggucgugcaauauuccccacuuaacgucaaugagcaaaagcuugccaguaucagauucgcaccugagcgucagggcccccgucaauucacugucucacgguucccgaagggccaugaugacuugagucgaguugcagacuucaccccagucaugaau

SEQ ID NO: 98 - RNA, 203 nt, Artificial Sequence
  Synthesized
caucaggcaguuucccagacauuacucacccguuauuagcuaccguuuccacgcggcauggcugcauccuuccuccccgcugaacaccuggaauucuacccccgccuucgccaccgguaucgaauuaaaccacauucugaaaacuuccgugggcgccauuguagcacgucgcacuuuaugaggucccgcagguuccccuacgg

SEQ ID NO: 99 - RNA, 203 nt, Artificial Sequence
  Synthesized
cuguuaccguucgacuugcauguguuaggccugcaucugggcacauccgagucuggaccgugucucauggcacggaguuagccgagcccggggauuucacaccaggguaucuaauccugcggccguacuccccagucacaacacgagcugacuccuccaguuuaucacuugauccacgauuacuagcccucccgaagguuaag

SEQ ID NO: 100 - DNA, 150 bp, Artificial Sequence; identical to SEQ ID NO: 90
  Synthesized

SEQ ID NO: 101 - DNA, 150 bp, Artificial Sequence
  Synthesized
tcttcactttaactcacactaccccacatccccctcctactcaaaaactcccctcccacaacccaaacccaccacaccaacaacaaccaaaactcacccaccccccctcactaacccaaaaaaaaaacaccaccaaccaaccataactac

SEQ ID NO: 102 - DNA, 150 bp, Artificial Sequence
  Synthesized
tcaattacctctctctcatctcacctcctacaccacaccctcccaactaaccacacaccaacacactcaaacttctctctatccctcccaatcaccctactccgcgccttccccttcccttcagtttcccctttttctctctttcttcac

SEQ ID NO: 103 - RNA, 45 nt, Artificial Sequence
  Synthesized
  misc_feature (1)..(1): Cholesterol TEG
uauagucaccagaaucgacgcuaaggagcucuagcggaacguaag

SEQ ID NO: 104 - RNA, 45 nt, Artificial Sequence
  Synthesized
  misc_feature (1)..(1): Cholesterol TEG
ggcaacguucuacuuaacuucuguguucggaacaggugugaccuc

SEQ ID NO: 105 - RNA, 45 nt, Artificial Sequence; identical to SEQ ID NO: 103
  Synthesized

SEQ ID NO: 106 - RNA, 45 nt, Artificial Sequence; identical to SEQ ID NO: 104
  Synthesized

SEQ ID NO: 107 - DNA, 33 bp, Artificial Sequence
  Synthesized
  misc_feature (1)..(1): Cholesterol TEG
tcaccagaatcgacgctaaggagctctagcgga

SEQ ID NO: 108 - DNA, 33 bp, Artificial Sequence
  Synthesized
  misc_feature (1)..(1): Cholesterol TEG
gttctacttaacttctgtgttcggaacaggtgt

SEQ ID NO: 109 - DNA, 33 bp, Artificial Sequence; identical to SEQ ID NO: 107
  Synthesized

SEQ ID NO: 110 - DNA, 33 bp, Artificial Sequence; identical to SEQ ID NO: 108
  Synthesized

(19) United States
(12) Patent Application Publication
(10) Pub. No.: US 2020/0339981 A1
(43) Pub. Date: Oct. 29, 2020

(54) SET OF ANTI-PATHOGENIC NUCLEIC ACIDS, COMPOSITIONS AND USES THEREOF

(71) Applicant: AUGMANITY NANO LTD., Rehovot (IL)

(72) Inventors: Anastasia SHAPIRO, Rishon Lezion (IL); Ido BACHELET, Tel-Aviv (IL); Almogit ABU-HOROWITZ, Herzliya (IL)

(21) Appl. No.: 16/930,879

(22) Filed: Jul. 16, 2020

Related U.S. Application Data
(63) Continuation-in-part of application No. PCT/IL2019/050068, filed on Jan. 17, 2019.
(60) Provisional application No. 62/618,107, filed on Jan. 17, 2018.

(51) Int. Cl. C12N 15/11; C12N 15/63; A61K 31/7105; A61K 31/711; A61P 31/04

(52) U.S. Cl. CPC C12N 15/11; C12N 15/63; A61P 31/04; A61K 31/711; A61K 31/7105

(57) ABSTRACT

The present invention provides to a set of anti-pathogenic nucleic acids capable of forming in cellulo one or more nucleic acid origami nanostructure together with one or more pathogenic RNA such as ribosomal RNA, pre-rRNA or mRNA present in the pathogenic cells. The present invention further provides nucleic acid constructs comprising the sequences of the set of anti-pathogenic staple nucleic acids and a vector comprising such a nucleic acid construct. Compositions, including pharmaceutical compositions, comprising the sets, the construct or the vector and use thereof are provided as well.

### FIELD OF THE INVENTION

The present invention provides sets of anti-pathogenic nucleic acids capable of forming in cellulo a nucleic acid origami nanostructure together with the pathogenic cellular RNA such as ribosomal RNA, pre-rRNA and mRNA compositions comprising said sets and uses thereof.

### BACKGROUND OF THE INVENTION

Many antibiotics are known to target ribosomal RNA (rRNA) in prokaryotes and thereby alter protein translation. Their diverse structures witness both to the importance attached to this class of drugs and to the long period of their development since their discovery. Concomitant with the marketing of powerful antibiotics has emerged the phenomenon of antibiotic resistance which poses a serious threat to human health. The recent slow-down in the pace of novel antibiotic development has further complicated the global health issue (Hong et al., Acta Pharmaceutica Sinica B 2014; 4(4):258-265).

Ribosomal RNA is the most commonly exploited RNA target for small molecules. The bacterial ribosome comprises 30S and 50S ribonucleoprotein subunits, contains a number of binding sites for known antibiotics and is an attractive target for novel anti-bacterial agents. The large difference between prokaryotic and eukaryotic rRNA enables rRNA-targeting against a broad spectrum of pathogenic bacteria.

Additional development in antibacterial treatment is antibacterial antisense oligonucleotides. This is generally described as RNA silencing in bacteria using synthetic nucleic acid oligomer mimetics to specifically inhibit essential gene expression and achieve gene-specific antibacterial effects. Usually the antibacterial antisense oligonucleotides are designed to bind the target mRNA to prevent translation or bind DNA to prevent gene transcription respectively (Bai and Luo., A Search for Antibacterial Agents, 2012, chapter 16: 319-344; ISBN 978-953-51-0724-8, InTech, Chapters).

DNA origami has emerged as a promising assembly technique in DNA nanotechnology with a broad range of applications (Rothemund, 2006, Nature 440, 297-302). Since the creation of this method, software was developed to assist the process using computer-aided design (CAD) software allowing pre-calculating and determining the sequences of scaffolds and staples needed to form a certain shape. In most cases, DNA origami technique is used for in vitro preparation of a DNA nanostructures Endo et al., (Chem. Eur. 1 2014, 20, 15330-15333) and Wang et al., (Chem. Commun., 2013, 49, 5462-5464). Gerasimova and Kolpashchikov (Angew. Chem. Int. Ed. Engl. 2013 52(40) doi:10.1002anie.201303919 described an assay that analyzes bacterial RNA exploiting deoxyribozyme sensors—two DNA stands containing fragments complementary to a target analyte and fragments complementary to a fluorophore and quiencher-labeled fluorogenic reporter substrate. Gerasimova and Kolpashchikov showed that hybridization of the staples to the RNA unwinds its secondary structure to form “deoxyribozymes-on-a-string” complex.

There is a clear and unmet need for a development of additional compositions exploiting novel antibacterial mechanisms.

### SUMMARY OF THE INVENTION

The present invention is based, in part, on the observation that a set of staple nucleic acids, capable of binding to bacterial rRNA and subsequently forming an origami nanostructure with it, was capable of inhibiting bacterial growth and had a bactericidal effect, when the staples were produced or introduced in the bacteria.

According to one aspect, the present invention provides a set of anti-pathogenic staple nucleic acids, wherein the set comprises a plurality of different staple nucleic acids that bind specifically to one or more pathogenic RNA molecules to form one or more nucleic acid origami nanostructure(s), wherein the RNA is selected from ribosomal RNA (rRNA) and mRNA. According to the teaching of the present invention, the origami nanostructure(s) are formed in cell, i.e. in cellulo. According to some embodiments, the pathogen is selected from bacteria, fungi and parasites. According to other embodiments, the staple nucleic acids are selected from DNA or RNA nucleic acids. According to some embodiments, the set comprises both DNA and RNA staples. Thus, in one embodiment, the present invention provides a set of anti-pathogenic staple nucleic acids capable of forming in cellulo one or more nucleic acid origami nanostructure(s) with a pathogenic RNA selected from rRNA, pre-rRNA and mRNA. According to another embodiment, the present invention provides a set of anti-pathogenic staple nucleic acids forming in cellulo one or more nucleic acid origami nanostructure(s) with a pathogenic RNA.

According to some embodiments, the RNA is a pathogenic rRNA, such as bacterial rRNA. According to one embodiment, the set of staples comprises staple nucleic acids having the sequences SEQ ID NOs: 2-54. According to another embodiment, the set comprises nucleic acids having the sequences SEQ ID NOs: 55-81. According to one embodiment, the set of staples comprises from 10% to 90%, from 20% to 80%, from 30% to 70% or from 40% to 60% of staples of the set having nucleic acid sequences SEQ ID NOs: 2-54 or of a set having nucleic acid sequences SEQ ID NOs: 55-81. According to a further embodiment, the set of staples comprises staple nucleic acids having the sequences SEQ ID NO: 82-85.

According to some embodiments, the RNA is a pathogenic mRNA, such as bacterial mRNA. According to one embodiment, the set of staples comprises staples that bind to at least 2 different mRNA molecules.

According to some embodiments, the staples are membrane-permeable staples. According to one embodiment, the staples are selected to be membrane-permeable.

According to some embodiments, the staples of the present invention are operably linked to at least one of a promoter, operator and terminator. According to some embodiments, some of the staples are operably linked to at least one of a promoter, operator and terminator. According to one embodiment, each one of the staples is operably linked to at least one of a promoter, operator and/or terminator. According to some embodiments, the RNA is a bacterial rRNA and the set comprises staples having SEQ ID NOs: 86-89 or SEQ ID NOs: 92-95.

According to some embodiments, the staples of the present invention are conjugated to a permeability-enhancing moiety.

According to some embodiments, the anti-pathogenic staple nucleic acids of the set of the present invention form a nucleic acid origami nanostructure with one or more pathogenic RNA molecules in vivo, e.g. in bacteria.

According to some embodiment, the set of anti-pathogenic staples is formulated in a delivery system vehicle, According to some embodiments, the vehicle is selected from liposomes, micelles, nanoparticle, viral nanoperticals, carbonano tubes and any other known vehicles. According to some embodiments, the delivery system vehicle allows or helps internalization of the set of staples into the cell.

According to another aspect, the present invention provides a nucleic acid construct comprising the sequences of the staple nucleic acids of the set of the present invention. According to some embodiments, the nucleic acid construct comprises a spacer between each pair of staple sequences. According to some embodiments, the spacer is a cleavable spacer. According to one embodiment, the spacer has a nucleic acid sequences selected from SEQ ID NOs: 100, 101 and 102. According to some embodiments, the nucleic acid construct comprises sequence SEQ ID NO: 91. According to one embodiment, the set of staples of the present invention are obtained upon transcription of the nucleic acid construct and optionally further splicing of the resulted RNA molecule in cellulo. According to one embodiment, the set of staples of the present invention are obtained upon separated transcription of each one of the staples encoded by the construct. According to some embodiments, the transcription occurs in vivo, e.g. in bacteria. According to one embodiment, the nucleic acid construct is conjugated with permeability-enhancing moiety.

According to another aspect, the present invention provides a vector comprising the nucleic acid construct or a set of staples of the present invention. According to some embodiments, the vector is selected from a plasmid, phage, expression vector, cosmid, and artificial chromosome. According to one embodiment, the vector is a plasmid. According to one embodiment, the set of staples of the present invention are obtained upon transcription of the vector or of the nucleic acid construct located within the vector and optionally further splicing or cleavage of the resulted RNA molecule. According to one embodiment, the set of staples of the present invention are obtained upon separated transcription of each one of the staples encoded by the vector. According to some embodiments, the transcription is occurred in vivo, e.g. in bacterial. According to one embodiment, the vector is conjugated with permeability-enhancing moiety. According to one embodiment, the vector is formulated in a delivery system vehicle

According to another aspect, the present invention provides a method for treating a pathogen comprising contacting the pathogen with the set of anti-pathogenic staple nucleic acids or with the nucleic acid construct or with a vector of the present invention. According to some embodiments, the method comprises transforming, transfecting or infecting the pathogen.

According to a further aspect, the present invention provides a composition comprising a plurality of sets of anti-pathogenic nucleic acids of the present invention, or a plurality of nucleic acid constructs of the present invention or a plurality of vectors of the present invention. According to some embodiments, the composition is a pharmaceutical composition. According to one embodiment, the pharmaceutical composition of the present invention is for use in treating a pathogenic infection. According to one embodiment, the pathogenic infection is a bacterial infection. According to one embodiment, the present invention provides a pharmaceutical composition for use in treating bacterial infection, wherein the pharmaceutical composition comprises a plurality of sets of anti-pathogenic nucleic acids, wherein said set comprises staple nucleic acid molecules selected from SEQ ID NOs: 2-54, SEQ ID NOs: 55-81, SEQ ID NOs: 82-85, SEQ ID NOs: 86-89 and SEQ ID NOs: 92-95. According to some embodiments, wherein the set comprise from 10% to 90%, from 30 to 80% or from 40 to 60% of staples of a set of staple nucleic acid molecules selected from SEQ ID NOs: 2-54, SEQ ID NOs: 55-81, SEQ ID NOs: 82-85, SEQ ID NOs: 86-89 and SEQ ID NOs: 92-95. According to another embodiment, the pharmaceutical composition for use in treating bacterial infection comprises a plurality of constructs or a plurality of vectors comprising a set of staple nucleic acids selected from SEQ ID NOs: 2-54, SEQ ID NOs: 55-81, SEQ ID NOs: 82-85, SEQ ID NOs: 86-89 and SEQ ID NOs: 92-95, or from 10% to 90%, from 30 to 80% or from 40 to 60% of staples of said set. According to some embodiments, the pharmaceutical composition for use in treating bacterial infection comprises a plurality of constructs having SEQ ID NOs: 91. According to one embodiment, the pharmaceutical composition for use in treating bacterial infection comprises a plurality of vectors comprising nucleic acid sequence SEQ ID NOs: 91.

According to yet another aspect, the present invention provides a method of treating a pathogenic infection in a subject in a need thereof comprising administering to said subject a therapeutically effective amount of the sets of staple nucleic acids of the present invention or the pharmaceutical composition of the present invention. According to one embodiment, the pathogenic infection is a bacterial infection.

### BRIEF DESCRIPTION OF FIGURES

FIG. 1 shows AFM images of the 16S rRNA-DNA structures obtained using E. coli 16S rRNA as a scaffold, staples having SEQ ID NOs: 2-54, folded using protocol 1 and further incubated at 37° C. for 5.5 days.

FIG. 2 shows AFM images of the 16S rRNA-DNA structures obtained using E. coli 16S rRNA as a scaffold, staples having SEQ ID NOs: 55-81, folded using protocol 1 and further incubated at 37° C. for 5.5 days.

FIG. 3 shows growth curves of E. coli transformed or not with a vector comprising a set of 4 staples (SEQ ID NO: 86-89) in two different IPTG concentrations 0 mM IPTG—FIG. 3A and 1 mM IPTG—FIG. 3B.

FIG. 4 shows growth of E. Coli either transfected or not a vector comprising a set of 4 staples (SEQ ID NO: 86-89) on LB plates with kanamycin 50 mM.

FIG. 5 shows the validation of presence of 4 staples in transfected cells.

FIG. 6 shows the effect of RNA staples targeting 5S rRNA on growth of Staphylococcus aureus.

### DETAILED DESCRIPTION OF THE INVENTION

The present invention is based on the observation that administering to bacteria a set of nucleic acids capable of forming a nucleic acid origami nanostructure together with bacterial ribosomal RNA provides a bactericidal effect and inhibits bacterial growth.

The present invention in one aspect provides a set of anti-pathogenic staple nucleic acids, wherein the set comprises a plurality of different staple nucleic acids that bind specifically to one or more pathogenic RNA to form one or more nucleic acid origami nanostructure(s). According to one embodiment, the origami nanostructure(s) is formed in cellulo. According to one embodiment, the pathogenic RNA is a cellular RNA. According to one embodiment, the RNA is selected from ribosomal RNA (rRNA), pre-rRNA and messenger RNA (mRNA). Thus, in one embodiment, the present invention provides a set of anti-pathogenic staple nucleic acids capable of forming one or more nucleic acid origami nanostructure(s) with one or more pathogenic cellular RNA in cellulo. Examples of the pathogenic cellular RNA are pathogenic rRNA and pathogenic mRNA. According to one embodiment, the set is capable of forming origami nanostructure(s) with more than one scaffold. Thus, in one aspect, the present invention provides a set of anti-pathogenic staple nucleic acids, wherein the set comprises plurality of different staple nucleic acids that bind specifically to one or more pathogenic RNA to form in cellulo nucleic acid origami nanostructure(s), wherein the RNA is selected from ribosomal RNA (rRNA) and messenger RNA (mRNA).

The term “nucleic acid” as used herein refers to a sequence (polymer) of deoxyribonucleotides or ribonucleotides. In addition, the nucleic acid includes analogues of natural polynucleotides, unless specifically mentioned. The nucleic acid may be selected from deoxyribonucleic acid (DNA), ribonucleic acid (RNA), and analogues thereof, but is not limited thereto. The term encompasses DNA and RNA, either single stranded or double stranded and chemical modifications thereof. The term encompasses also peptide nucleic acid (PNA) and locked nucleic acid (LNA),

The term “polynucleotide” as used herein refers to a long nucleic acid comprising more than 200 nucleotides.

The term “oligonucleotide” as used herein refers to a short single stranded or double stranded sequence of nucleic acid such as ribonucleic acid (RNA), deoxyribonucleic acid (DNA) or mimetics thereof, said nucleic acid has typically less than or equal to 200 nucleotides.

The terms “nucleotide base”, “nucleotide” and “nucleic acid base” are used herein interchangeably and refer to a DNA or RNA base and any modification thereof.

The term “pathogen” and “pathogenic” are used herein interchangeably and refer to bacteria, fungi, parasites, and other microorganisms capable of exerting pathogenic effects in multicellular organisms. Thus, the use of the term “pathogen” contemplates microorganisms capable of causing disease in plants, mammals, including humans. The term “anti-pathogenic” refers to a compound(s) such as set of staples that act against a pathogen, e.g. kills, inhibits or prevents growth, spread division, fission and replication. According to some embodiments, the pathogen is selected from a bacteria, fungi and parasites.

The terms “in cellulo” and “in vivo” are used herein interchangeably and have the meaning of “in a living cell”. According to the teaching of the present invention, the origami nanostructure is formed within the living cell, e.g. in a bacterial cell.

The terms “scaffold”, “scaffold strand”, and “scaffold nucleic acid” are used herein interchangeably and refer to a long nucleic acid strand. Examples of such nucleic acids are rRNA, mRNA, and pre-rRNA, and in particular bacterial rRNA and bacterial mRNA polynucleotide strand. According to one embodiment, the scaffold is a parasitic rRNA. According to a further embodiment, the scaffold is a fungal rRNA. According some embodiments, the scaffold or a fragment or a region of the scaffold is a single stranded nucleic acid, a double stranded nucleic acid that unfolds and refolds upon binding of a staple nucleic acid to form a desired structure or a mixture thereof.

The terms “staple”, “staple strand”, “staple nucleic acid” and “staple nucleic acid molecule” are used herein interchangeably and refer to single stranded nucleic acid designed to hybridize with a pathogenic RNA in particular to hybridize with at least two non-contiguous sequences of the pathogenic RNA. According to some embodiments, the staple is designed to hybridize with a pathogenic rRNA. According to other embodiments, the staple is designed to hybridize with a pathogenic mRNA. According to one embodiment, the pathogen is bacteria. The sequences of the staple nucleic acids are designed to hybridize with at least two non-contiguous sequences of the scaffold nucleic acid and therefore to force its folding into a particular shape. According to some embodiments, the staples are synthetic nucleic acids. According to some embodiments, the staples are oligonucleotides. According to another embodiment, the staples are polynucleotides. Hybridization of the staples nucleic acids with two or more non-contiguous fragments of a scaffold forces juxtaposing these fragments and therefore forces the scaffold to fold to a particular 2D or 3D structure. According to some embodiments, the staple is devoid of non-binding regions. According to one embodiment, all bases of the staple form bonds with the scaffold. According to some embodiments, the staple nucleic acids has a non-binding region between the two binding regions. According to one embodiment, the non-binding region between the two binding regions of the staple is shorter than the fragment between the two non-contiguous fragments of a scaffold to which the staple binds. According to one embodiment, the non-binding region of the staple is shorter 1.5, 2, 3, 4, or 5 times than the fragment between the two non-contiguous fragments of a scaffold. According to one embodiment, the non-binding region of the staple consists of 2, 3, 4, 5, or 6 nucleic acids. According to some embodiments, the staple comprise a non-binding region on one or on two of its termini. According to some embodiments, the staples are DNA nucleic acids (DNA staples). According to some embodiments, the staples are RNA nucleic acids (RNA staples). According to one embodiment, the staple nucleic acid hybridizes with at least two non-contiguous sequences of rRNA, pre-rRNA or mRNA. According to some embodiments, the staple binds to two different scaffolds simultaneously, subsequently such staple forces juxtaposing said two scaffolds. According to another embodiment, the staple nucleic acids bind specifically to a bacterial rRNA, pre-rRNA or mRNA. In a further embodiment, the staple nucleic acids bind specifically to a parasitic rRNA, pre-rRNA or mRNA. According to another embodiment, the staple nucleic acids bind specifically to a fungal rRNA, pre-rRNA or mRNA. The term “anti-pathogenic staple” as used herein means that the staple binds to a pathogenic RNA, e.g. pathogenic rRNA, pre-RNA, or mRNA. According to a further embodiment, the term “anti-fungal staple” as used herein refers to staple that binds to a fungi RNA, e.g. fungi rRNA or mRNA. According to some embodiments, the staple is PEGylated. According to some embodiments a staple comprises modified nucleotides such as PEGilated nucleotides, LNA, 2-MethoxyEthoxy, 2′-O-Methyl, 2-aminopurine, deoxyUridine, Fluor bases and inverted bases. Other contemplated modifications are biotin binding, methylation, phosphorylation, SS, NH2, COOH, Thiol, Azide, Alkynes, NSH attachment of fluorophores, quencher, etc. According to some embodiments, a staple is a conjugated staple. According to some embodiments, a staple is conjugated to a cholesterol, TEG, PEG, Digoxigenin, I-Linker, polymeric spacer, protein, peptide, Polysaccharides etc. According to some embodiments, the modified nucleotide is an inverted dT. According to some embodiments, the inverted dT is located at a terminus or at both termini of the nucleic acid. According to some embodiments, a staple is a conjugated staple. According to another embodiment, the nucleic acid is conjugated with cholesterol. According to another embodiment, the nucleic acid is conjugated with cholesterol-TEG. According to some embodiments, the staple nucleic acids has at least 99%, 98%, 97%, 96% or 95% sequence complementarity to each fragment of the scaffold nucleic acid to which it designed to bind. According to other embodiments, the staple nucleic acids has at least 50%, 55%, 60%, 65%, 70%, 75%, 80%, 85%, 90%, 95% or 99% sequence complementarity to each fragment of the scaffold nucleic acid to which it binds. As referred to herein, the term “complementary” is directed to base pairing between strands of nucleic acids. As known in the art, each strand of a nucleic acid may be complementary to another strand in that the base pairs between the strands are non-covalently connected via two or three hydrogen bonds. For example, “100% complementarity” indicates that all the nucleotides in each strand form base pairs with the complement strand. For example, “95% complementarity” indicates that 95% of the nucleotides in each strand form base pair with the complement strand. The term sufficient complementarity may include any percentage of complementarity from about 30% to about 100%.

The terms “set of staples” and “set of staple nucleic acids” are used herein interchangeably and refer to a set of staple nucleic acids, i.e. 2 or more different staple nucleic acids, configured to, used for or capable of folding a nucleic acid origami nanostructure(s) based on particular scaffold(s), such as bacterial RNA, e.g. bacterial rRNA, pre-rRNA, or mRNA. In some embodiments, the set of staples may comprise a single staple capable of folding an origami structure. Nevertheless, in some specific embodiments, the set comprises one staple. The term “antibacterial staple” as used herein means that the staple that binds to a bacterial RNA such as rRNA, pre-RNA, or mRNA. According to some embodiments, the set of staples further comprises nucleic acids complementary to a fragment or a region of a scaffold. The terms “nucleic acid origami nanostructure”, “origami nanostructure”, “nanostructure” and “RNA origami nanostructure” are used herein interchangeably and refer to a 2 dimensional or 3 dimensional custom shaped nanostructure comprising pathogenic RNA as a scaffold, and a plurality of staples nucleic acids, wherein said pathogenic RNA is folded by said staples to a desired and/or designed 2D or 3D nanostructure. According to one embodiment, the pathogenic RNA is pathogenic rRNA. According to another embodiment, the pathogenic RNA is pathogenic mRNA. According to another embodiment, the pathogenic RNA is pathogenic pre-rRNA. According to some embodiments, the pathogen is selected from a bacteria, fungi and parasites. According to some embodiments, the nucleic acid origami nanostructure may comprise 2 or more scaffolds. Thus, according to one embodiments, a set of staples comprises staples that bind to one scaffold and form one origami nanostructure. According to other embodiments, the set may comprises staples that bind to two or more different scaffolds and form two different origami structures. According to yet another embodiment, the set of staples may comprise staples that bind to two different scaffolds to fold one origami nanostructure. According to some embodiments, the set of scaffold may comprise combinations of abovementioned sets. According to any one of the above embodiments, the staples of the set of staples are configured to form nucleic acid origami nanostructure in a pathogenic RNA in cellulo.

According to some embodiments, the set of staple nucleic acids comprises from 2 to 10000 different staple nucleic acids from 2 to 5000 different staple nucleic acids or from 2 to 1000. According to some embodiments, the set of staple nucleic acids comprises from 2 to 400 different staple nucleic acids. According to some embodiments, the set comprises from 5 to 250, from 10 to 200, from 15 to 180, from 20 to 150, from 25 to 120, from 30 to 100, from 35 to 80, from 40 to 60 staple nucleic acids. According to some embodiments, the set comprises from 2 to 10 different staples. According to one embodiment, the set comprises from 3 to 6 different staples. According to another embodiment, the set comprises 2, 3, 4, 5, 6, 7, 8, 9 or 10 different staples. According to one embodiment, the set comprises from 10 to 30 different staples. According to one embodiment, the set comprises 1 staple.

The term “different” with respect to nucleic acid means that the nucleic acids have different sequences, e.g. having less than 70% sequence identity, or less than 80% sequence identity or less than 60% sequence identity.

According to one embodiment, the set of staple comprises DNA staples. According to another embodiment, the set of staples comprises RNA staples. According to a further embodiment, the staple nucleic acids are a combination of RNA and DNA nucleic acids

According to some embodiments, the staple nucleic acids consist of from 5 to 120 or from 10 to 100 nucleotides. According to one embodiment, consist of from 5 to 80 nucleotides. According to another embodiment, the staple oligonucleotides consist of from 6 to 60, from 7 to 50 or from 8 to 45 nucleotides. According to a further embodiment, the staple oligonucleotides consist of from 10 to 45 or from 15 to 40 nucleotides. According to certain embodiment, the staple oligonucleotides consist of from 7 to 75 nucleotides. According to another embodiment, the staple consists of from 10 to 300 nucleotides, from 15 to 280, from 20 to 250, from 25 to 220, from 30 to 200, from 35 to 180, from 40 to 150, from 50 to 120, or from 70 to 100 nucleotides. In some embodiments, the staples consist of from 100 to 600, from 120 to 560, from 150 to 520 from 200 to 480, from 250 to 420, from 300 to 380 or from 320 to 360 nucleotides. According to some embodiments, the nucleic acid staples consist of from 200 to 400 nucleotides. In other embodiments, the staple consists of up to 60% of the number of nucleotides in the target scaffold. In other embodiments, the staple consists of up to 30%, 40% or up to 50% of the number of nucleotides in the target scaffold.

As discussed above, generation of RNA origami structures involves folding single stranded scaffold nucleic acid strand(s) into a particular shape using a plurality of rationally designed staple nucleic acids. The scaffold and the staples form a double stranded nucleic acid. According to any one of the above embodiments, the scaffold and the staples of the present invention form a double helix having A-conformation, e.g. A-RNA:RNA or A-RNA:DNA conformation which is similar to A-DNA double helix geometry and have similar structural properties of A-DNA double helix.

According to some embodiments, the staples are RNA nucleic acid staples. According to one embodiment, the scaffold and the RNA nucleic acid staples form A-conformation double helix. The term “A-conformation” as used herein refers to a polynucleotide double helix having the characteristics similar to that of A-DNA double helix. Therefore, the terms “A-RNA:RNA”, “A-RNA” are used herein interchangeably and refers to RNA-RNA double helix having A-conformational geometry (A-type helix), and the terms “A-RNA:DNA” and “A-DNA”, are used herein interchangeably and refer to RNA-DNA double helix having A-conformational geometry. According to one particular embodiment, the scaffold and the RNA staples form A-RNA:RNA double helix having periodicity of 12 base-pairs per turn. According to one particular embodiment, the scaffold and the RNA staples form A-RNA:RNA double helix having periodicity of 11 base-pairs per turn. The terms “base-pairs per turn” and “nucleotides per turn” are used interchangeably. The term “nucleotides per turn” refers to number of nucleotides of a single stranded scaffold nucleic acid in the double stranded structure found in one turn of the helix. According to one embodiment, the nucleic acid staples are designed to form A-RNA:RNA double helix with a scaffold. According to one embodiment, the RNA-RNA double helix has A-conformational geometry. According to another embodiment, the present invention provides a set of staples that form RNA origami with one or more pathogenic rRNA molecules to form a nanostructure comprising one scaffold rRNA nucleic acid strands and a plurality of staple of the present invention or staples transcribed from the nucleic acid sequences of the staples of the present invention, wherein the scaffold and the staples form an RNA-RNA double helix.

According to any one of the aspects and embodiments of the invention, the terms “nucleic acid comprising the nucleic acid sequence as SEQ ID NO: X”, “nucleic acid comprising the sequence SEQ ID NO: X”, “nucleic acid comprising SEQ ID NO: X” and “nucleic acid having SEQ ID NO: X” are used herein interchangeably. The terms “nucleic acid consisting of the nucleic acid sequence SEQ ID NO: X”, “nucleic acid consisting of sequence SEQ ID NO: X” “nucleic acid consisting of SEQ ID NO: X” and “nucleic acid of SEQ ID NO: X” are used herein interchangeably. In any one of the aspects and embodiments of the invention the term “comprise” encompasses also the term “consist” therefore in any one of the aspects and embodiments of the invention the term “nucleic acid comprising the sequence SEQ ID NO: X” encompasses the term “nucleic acid consisting of sequence SEQ ID NO: X” and may be replaced by it.

According to some embodiments, the set of staples is designed and/or configured to bind to bacterial rRNA. According to some embodiments, the set of staples is designed and/or configured to bind to fungal rRNA. According to some embodiments, the set of staples is designed and/or configured to bind to parasite rRNA.

According to some embodiments, the pathogenic RNA is a ribosomal RNA (rRNA). According to one embodiment, the pathogenic rRNA is a bacterial rRNA. According to one embodiment, the bacterial rRNA is selected from 16S, 23S, 5S rRNA and pre-rRNA. The term “pre-rRNA”, also known as a primary transcript, refers to a bacterial ribosomal RNA precursor which typically containing up to 3 copies of each one of 16S, 23S, and 5S rRNA, or to any other precursor of rRNA. According to other embodiments, the pathogenic rRNA is a fungal or parasitic rRNA. According to one embodiment, the pathogenic rRNA is selected from 18S, 25S, 26S, 28S, 5.8S rRNA.

According to one embodiment, the pathogenic RNA is rRNA. According to one embodiment, the present invention provides a set of anti-pathogenic staple nucleic acids, wherein the set comprises a plurality of different staple nucleic acids that bind specifically to pathogenic rRNA molecules to form nucleic acid origami nanostructure(s). According to one embodiment, the set comprises staples that bind to one pathogenic rRNA to form one origami nanostructure. According to another embodiment, the set of staples bind to two or more identical rRNA molecules to form one or more origami nanostructures. According to yet another embodiment, the set of staples bind to two or more different rRNA molecule to form one of more different origami nanostructures. According to one embodiment, the pathogenic rRNA is a bacterial rRNA. According to one embodiment, the set of staples designed to bind bacterial rRNA comprises staple nucleic acids having nucleic acid sequences SEQ ID NOs: 2-54. According to another embodiment, the set comprises staple nucleic acids having sequences SEQ ID NOs: 55-81. According to a further embodiment, the set comprises staple nucleic acids having sequences SEQ ID NOs: 82-85. According to a further embodiment, the set comprises staple nucleic acids having sequences from SEQ ID NOs: 2-54,SEQ ID NOs: 55-81, SEQ ID NOs: 82-85 and SEQ ID NOs: 92-95. According to one embodiment, the set of staples comprises from about 10% to about 99%, about 20% to about 95%, about 30% to about 90%, about 40% to about 85%, about 50% to about 80% or about 60% to about 70% of staples of a set of staples nucleic acids having the sequences SEQ ID NOs: 2-54, or SEQ ID NOs: 55-81. According to some embodiments, the set of staples comprises variants of staples nucleic acids having the sequences SEQ ID NOs: 2-54,SEQ ID NOs: 55-81, SEQ ID NOs: 82-85 and SEQ ID NOs: 92-95. The terms “homolog” “variant”, “DNA variant”, “sequence variant” and “polynucleotide variant” are used herein interchangeably and refer to a nucleic acid having at least 85% sequence identity to the parent nucleic acid. According to some embodiments, the variant has at least 90%, at least 95%, at least 98% or at least 99% sequence identity to the parent polynucleotide. According to some embodiments, the set of staples comprises from about 10% to about 99%, about 20% to about 95%, about 30% to about 90%, about 40% to about 85%, about 50% to about 80% or about 60% to about 70% of variants of staples selected from SEQ ID NOs: 2-54,SEQ ID NOs: 55-81, SEQ ID NOs: 82-85 and SEQ ID NOs: 92-95.

According to any one of the above embodiments, the present invention provides a set of staple nucleic acids wherein at least one of the staples is operably linked to at least one of a promoter, operator or terminator. According to some embodiments, at least 5% at least 10, at least 15% at least 20%, at least 25%, at least 30%, at least 35%, at least 40%, at least 45%, at least 50%, at least 55% at least 60% at least 65%, at least 70%, at least 75%, at least 80%, at least 85%, at least 90%, or at least 95% of the of the staples are operably linked to at least one of a promoter, operator and terminator. According to other embodiments, the present invention provides a set of staple nucleic acids wherein each one of the staples is operably linked to at least one of a promoter, operator and terminator. According to one embodiment, each one of the staples is operably linked to a promoter. According to another embodiment, each one of the staples is operably linked to a terminator. According to a further embodiment, each one of the staples is operably linked to an operator. According to certain embodiments, each one of the staples is operably linked to a promoter and terminator. According to other embodiments, each one of the staples is operably linked to a promoter, operator and terminator.

According to one embodiment, the set of staples comprises nucleic acid sequences SEQ ID NO: 86-89. According to another embodiment, the set of staples comprises nucleic acid sequences SEQ ID NO: 92-95.

According to one embodiment, the set comprises any combination of sets of wherein the set comprising staples having sequences SEQ ID NOs: 2-55, SEQ ID NOs: 56-81, SEQ ID NOs: 82-85, SEQ ID NOs: 92-95, and SEQ ID NOs: 86-89.

According to some embodiments, the set comprises a staples having sequences SEQ ID NOs: 107-108 or SEQ ID Nos: 109-110. According to some embodiments, the rRNA is 5S rRNA. According to some embodiments, the staples comprise inverted dT nucleotide(s). According to some embodiments, the set of staples comprises variants of staples nucleic acids having the sequences SEQ ID NO: 107-108 or SEQ ID Nos: 109-110.

According to one embodiment, the pathogenic RNA is a bacterial rRNA. According to some embodiments, the staples are RNA nucleic acid staples. In some embodiments, the set comprises RNA staples having RNA sequence corresponding to sequences selected from SEQ ID NOs: 2-55, SEQ ID NOs: 56-81, SEQ ID NOs: 82-85, SEQ ID NOs: 92-95, and SEQ ID NOs: 86-89, in which thymidine (T) is substituted with uridine (U). According to some embodiments, the set of staples comprises staple nucleic acids having sequences SEQ ID NO: 96-99.

According to some embodiments, the set comprises a staples having sequences SEQ ID NOs: 103-104 or SEQ ID Nos: 105-106. According to some embodiments, the rRNA is 5S rRNA. According to some embodiments, the staples comprise inverted dT nucleotide(s). According to some embodiments, the set of staples comprises variants of staples nucleic acids having the sequences SEQ ID NO: 103-104 or SEQ ID Nos: 105-106.

According to one embodiment, the set comprises staple nucleic acids having sequences SEQ ID NOs: 2-95. According to one embodiment, the set comprises staple nucleic acids, wherein said staples are RNA staples corresponding to sequences SEQ ID NOs: 2-95, in which T is substituted for U. According to another embodiment, the set comprises a combination of nucleic acids having sequences SEQ ID NOs: 2-95 and RNA staples having RNA sequences corresponding to SEQ ID NOs: 2-95. According to one embodiment, the set of staples comprises from about 10% to about 99%, about 20% to about 95%, about 30% to about 90%, about 40% to about 85%, about 50% to about 80% or about 60% to about 70% of staples of said sets. According to one embodiment, the set of staples comprises from about 10% to about 99%, about 20% to about 95%, about 30% to about 90%, about 40% to about 85%, about 50% to about 80% or about 60% to about 70% of staples of a set of staple nucleic acids having the sequences SEQ ID NOs: 2-95.

According to any one of the above embodiments, the nucleic acids of the set of staples are separate molecules. According to another embodiment, the nucleic acids are joined into one nucleic acid molecule.

According to one embodiment, the pathogenic RNA is a pathogenic mRNA. According to some embodiments, the pathogenic mRNA is a bacterial mRNA. Thus, in one embodiment, the present invention provides a set of anti-pathogenic staple nucleic acids, wherein the set comprises a plurality of different staple nucleic acids that bind specifically to one or more pathogenic mRNAs, to form nucleic acid origami nanostructure(s). According to one embodiment, the staples of the set bind to one mRNA. According to another embodiment, the set of staples comprises staples that bind simultaneously to 2 or more different bacterial mRNAs. According to one embodiment, the set comprises staples that bind to 3, 4 or 5 different mRNA. The term “bind simultaneously to 2 or more different bacterial mRNAs” encompasses states in which one staple bind to two different mRNAs and to such states in which each staple bind to one mRNA and the set of staple comprises at least two staples, each binding to 2 different mRNAs.

According to any one of the above embodiments, the staples of the present invention are membrane permeable staples. The term “membrane-permeable” refers to moieties such as staples capable of crossing the cell membrane and enter a living cell. According to one embodiment, the set of staples are selected to penetrate cell membrane. According to some embodiments, from 1% or from 10% to 100% from 20 to 90% from 30 to 80%, from 40 to 70%, from 40 to 60% of the set of staples are membrane-permeable staples. According to one embodiment, the pathogenic RNA is a bacterial mRNA, and the set of staple comprises from 1% or from 10 to 100% from 20 to 90% from 30 to 80%, from 40 to 70%, from 40 to 60% membrane-permeable staples. According to some embodiments, the pathogenic RNA is rRNA or pre-RNA.

According to any one of the above embodiments, the present invention provides a set of staples, wherein the staples are conjugated to permeability-enhancing moieties. According to any one of the above embodiments, the present invention provides a set of staples, wherein from 10 to 100% from 20 to 90% from 30 to 80%, from 40 to 70%, from 40 to 60% of staples are conjugated to permeability-enhancing moieties.

According to some embodiments, the staples are produced in cellulo to form the nucleic acid origami nanostructure(s). According to other embodiment, the staples are introduced or transformed to the pathogen and subsequently the nucleic acid origami nanostructures is formed.

According to another aspect, the present invention provides a nucleic acid construct comprising the sequences of the staple nucleic acids of the set of anti-pathogenic nucleic acids according to the teaching of the present invention as described above.

According to one embodiment, the present invention provides a nucleic acid construct comprising the nucleic acid sequences of the set of anti-bacterial staple nucleic acids according to the present invention as described above. According to one embodiment, the set comprises staples that bind specifically to a pathogenic rRNA. According to another embodiment, the set comprises staples that bind specifically to one or more pathogenic mRNA molecules.

According to some embodiments, the nucleic acid construct comprises the nucleic acid sequences of the set of anti-parasitic or anti-fungal staples according to the present invention as described above.

According to one embodiment, the nucleic acid construct comprises nucleic acid sequences of a set of staples comprising staple nucleic acids having SEQ ID NOs: 2-54. According to another embodiment, the set comprises staple nucleic acids having sequences SEQ ID NOs: 55-81. According to a further embodiment, the set comprises staple nucleic acids having sequences SEQ ID NOs: 82-85. According to one embodiment, the set of staples comprises from about 10% to about 99%, about 20% to about 95%, about 30% to about 90%, about 40% to about 85%, about 50% to about 80%, about 60% to about 70%, or about 70% to about 90% of staples of a set of staples nucleic acids having the sequences SEQ ID NOs: 2-55, or SEQ ID NOs: 55-81. According to a further embodiment, the set comprises staple nucleic acids having sequences SEQ ID NOs: 109-110.

According to some embodiments, the nucleic acid construct comprises the sequences of a set of staples, wherein at least one of the staples is operably linked to a promoter. According to one embodiment, the nucleic acid construct comprises the sequences of a set of staples, wherein at least one of staples is operably linked to a terminator. According to some embodiments, the nucleic acid construct comprises the sequences of a set of staples, wherein at least 10%, at least 20%, at least 30%, at least 40%, at least 50%, at least 60%, at least 70%, at least 80%, at least 90% of staples is operably linked to a promoter and/or a terminator. According to certain embodiments, the nucleic acid construct comprises the sequences of a set of staples, wherein each one of staples is operably linked to a promoter. According to some embodiments, the nucleic acid construct comprises the sequences of a set of staples, wherein each one of staples is operably linked to a terminator. According to a further embodiment, each one of the staples is operably linked to an operator. According to certain embodiments, each one of the staples is operably linked to a promoter and terminator. According to other embodiments, each one of the staples is operably linked to a promoter, operator and terminator. According to one embodiment, the set of staples comprises nucleic acid sequences SEQ ID NO: 86-89. According to another embodiment, the set of staples comprises nucleic acid sequences SEQ ID NO: 92-95. According to one embodiment, the nucleic acid comprises nucleic acid sequence SEQ ID NO: 91. According to another embodiment, the nucleic acid consists of nucleic acid sequence SEQ ID NO: 91. According to a further embodiment, the set comprises staple nucleic acids having sequences SEQ ID NOs: 109-110. According to some embodiments, the nucleic acid construct comprises a spacer between every pair of staples' sequences. The term “spacer” as used herein refers to a nucleic acid sequence, either RNA or DNA, connecting two sequences of staples in a nucleic acid construct. According to one embodiment, the spacer is a cleavable nucleic acid sequence. According to one embodiment, the cleavable sequence is a hairpin-forming sequence. According to some embodiments, the hairpin is an enzymatically cleavable hairpin. According to some embodiments, the spacer has a nucleic acid sequence selected from SEQ ID NOs: 100, 101 and 102. According to any one of the above embodiments, staple nucleic acids are obtained upon transcription of the nucleic acid construct in cellulo. According to some embodiments, the staple nucleic acids are obtained upon transcription and further splicing of the obtained RNA molecule. It is understood that upon transcription of the nucleic acid RNA molecules are obtained. Therefore, the present invention provides RNA nucleic acid staples having RNA nucleic acid corresponding to sequence sequences selected from SEQ ID NOs: 86-89, SEQ ID NOs: 92-95, SEQ ID NO: 86-89 and SEQ ID NO: 92-95, in which thymidine is replaced by uridine. According to some embodiments, the nucleic acid construct is operably linked to an origin of replication. According to one embodiment, the set comprises staple nucleic acids having sequences SEQ ID NOs: 2-95. According to one embodiment, the set comprises staple nucleic acids, wherein said staples are RNA staples corresponding to sequences SEQ ID NOs: 2-95, in which T is substituted for U. According to a further embodiment, the set comprises staple nucleic acids having sequences SEQ ID NOs: 105-106. According to another embodiment, the set comprises a combination of nucleic acids having sequences SEQ ID NOs: 2-95 and RNA staples having nucleic acids sequences corresponding to SEQ ID NOs: 2-95. According to one embodiment, the set of staples comprises from about 10% to about 99%, about 20% to about 95%, about 30% to about 90%, about 40% to about 85%, about 50% to about 80% or about 60% to about 70% of staples of said sets. According to one embodiment, the set of staples comprises from about 10% to about 99%, about 20% to about 95%, about 30% to about 90%, about 40% to about 85%, about 50% to about 80% or about 60% to about 70% of staples of a set of staple nucleic acids having the sequences SEQ ID NOs: 2-95.

The term “promoter” as used herein refers to a regulatory sequence that initiates transcription of a downstream nucleic acid. The term “promoter” refers to a DNA sequence within a larger DNA sequence defining a site to which RNA polymerase may bind and initiate transcription. A promoter may include optional distal enhancer or repressor elements. The promoter may be either homologous, i.e., occurring naturally to direct the expression of the desired nucleic acid, or heterologous, i.e., occurring naturally to direct the expression of a nucleic acid derived from a gene other than the desired nucleic acid. A promoter may be constitutive or inducible. A constitutive promoter is a promoter that is active under most environmental and developmental conditions. An inducible promoter is a promoter that is active under environmental or developmental regulation, e.g., upregulation in response to xylose availability. Promoters may be derived in their entirety from a native gene, may comprise a segment or fragment of a native gene, or may be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. It is further understood that the same promoter may be differentially expressed in different tissues and/or differentially expressed under different conditions.

As used herein, the term “terminator” or “chain terminator” is meant to refer to a nucleic acid sequence that effectively terminates transcription.

The term “operator” as used here refers to a nucleic acid sequence to which a repressor binds to prevent or inhibit transcription from an associated promoter.

The terms “operably linked”, “operatively linked”, “operably encodes”, and “operably associated” are used herein interchangeably and refer to the functional linkage between a promoter and nucleic acid sequence, wherein the promoter initiates transcription of RNA corresponding to the DNA sequence. Thus in one embodiment, each one of the staple nucleic acids is operably linked to a promoter and to a termination site such that each staple nucleic acid is separately transcribed by the DNA transcription machinery, such as bacterial transcription machinery.

According to some embodiments, the nucleic acid construct comprises reverse sequences of the staple nucleic acids of the set of antibacterial staples according to the present invention as described above. According to other embodiments, the nucleic acid construct comprises reverse sequences of the staple nucleic acids of the set of anti-fungal or anti-parasitic staples according to the present invention as described above.

According to a further embodiment, the nucleic acid construct is conjugated to a permeability-enhancing moiety.

The term “permeability-enhancing moiety” refers to any moiety known in the art to facilitate actively or passively or enhance permeability of the compound into the cells. According to one embodiment, the permeability-enhancing moiety is a polysaccharide. According to one embodiment, the permeability-enhancing moiety are peptides or aptamers. According to other embodiments, the permeability-enhancing moiety is a hydrophobic entity. According to some embodiments, the permeability-enhancing moiety is cholesterol or a derivative thereof or inverted dT.

According to a further aspect, the present invention provides a vector comprising the nucleic acid construct of the present invention. According to one embodiment, the vector comprises origin of replication site.

According to one embodiment, the vector comprises a nucleic acid construct comprising the sequences of the staple nucleic acids of the set according to the present invention as described above. According to one embodiment, each one of the sequences of the staple nucleic acids is operably linked to a promoter and/or a termination site. According to some embodiments, the each one of the sequences of the staple nucleic acids is operably linked to an operator. According to another embodiment, the vector comprises a nucleic acid construct comprising the sequences of the staple nucleic acids of the set according to the present invention as described above, wherein the nucleic acid construct is operably linked to a promoter and to a termination site, and wherein the nucleic acid construct comprises a spacer between every pair of staples' sequences. According to some embodiments, the nucleic acid construct is operably linked to an origin of replication. According to one embodiment, the spacer is a cleavable nucleic acid sequence. According to one embodiment, the cleavable sequence is a hairpin-forming sequence. According to some embodiments, the hairpin is an enzymatically cleavable hairpin.

According to some embodiments, the nucleic acid construct comprising the sequences of the staple nucleic acids is transcribed by the DNA transcription machinery, such as bacterial transcription machinery, and consequently spliced or parsed either via self-splicing or via enzymatic splicing to produce RNA staple nucleic acids. According to some embodiments, the cleavable sequence is cleavable by or consists of ribozyme.

According to some embodiments, the vector comprises nucleic acid sequences of a set of staples having sequences SEQ ID NOs: 2-54. According to some embodiments, the vector comprises nucleic acid sequences of a set of staples having sequences SEQ ID NOs: 55-81. According to other embodiments, the vector comprises nucleic acid sequences of a set of staples having sequences SEQ ID NO: 82-85. According to another embodiment, the vector comprises 10% to 90% of staples of a set of staple nucleic acids having the sequences SEQ ID NOs: 2-54 or SEQ ID NOs: 55-81. According to one embodiment, the vector comprises sequences of staples having sequences SEQ ID NO: 82-85. According to another embodiment, the vector comprises sequences of staples having sequences SEQ ID NO: 86-89. According to a further embodiment, the vector comprises sequences of staples having sequences SEQ ID NOs: 92-95. According to a further embodiment, the vector comprises sequences of staples having sequences SEQ ID NOs: 109-110. According to any one of the embodiment, the staple nucleic acids are obtained upon transcription of the vector. According to some embodiments, the staple nucleic acids are obtained upon transcription and further splicing of the obtained RNA molecule.

According to one embodiment, the vector comprises a nucleic acid construct comprising the reverse sequences of the staple nucleic acids of the set according to the present invention as described above. According to one embodiment, each one of the reverse sequences of the staple nucleic acids is operably linked to a promoter. According to such embodiments, each reverse sequences of staple nucleic acid is separately transcribed by the transcription machinery to produce RNA staple nucleic acid.

According to another embodiment, the vector comprises a nucleic acid construct comprising the reverse sequences of the staple nucleic acids of the set according to the present invention as described above, wherein the nucleic acid construct is operably linked to a promoter and terminator, and wherein the nucleic acid construct encodes for a spacer between every pair of staples' sequences or a cleavable sequence between every pair of staples' sequences. According to one embodiment, the spacer is a cleavable nucleic acid sequence. According to another embodiment, the cleavable sequence is a hairpin-forming sequence. According to some embodiments, the hairpin is an enzymatically cleavable hairpin. According to some embodiments, the spacer has a nucleic acid sequence selected from SEQ ID NOs: 100, 101 and 102. According to such embodiments, the nucleic acid comprising the sequences of the staple nucleic acids is transcribed by the transcription machinery and consequently might be spliced or parsed either via self-splicing or via enzymatic splicing to obtain RNA staple nucleic acids. According to some embodiments, the cleavable sequence is cleavable by or consists of ribozyme. According to some embodiments, the vector comprises an origin of replication.

The terms “vector” and “expression vector” are used herein interchangeably and refer to any viral or non-viral vector such as plasmid, virus, retrovirus, bacteriophage, cosmid, artificial chromosome (bacterial or yeast), phage, phagemid, binary vector in double or single stranded linear or circular form, or nucleic acid, sequence which is able to transform host cells and optionally capable of replicating in a host cell. The vector may contain an optional marker suitable for use in the identification of transformed cells, e.g., tetracycline resistance, kanamycin resistance or ampicillin resistance. A cloning vector may or may not possess the features necessary for it to operate as an expression vector. According to one embodiment, the expression vector comprises an origin of replication.

According to one embodiment, the vector is a plasmid. According to another embodiment, the vector is a phage, e.g. bacteriophage. According to another embodiment, the vector is an expression vector. According to any one of the embodiment, staple nucleic acids are obtained upon replication or transcription of the vector.

According to a further embodiment, the vector is conjugated to a permeability-enhancing moiety.

According to any one of the embodiments of the present invention, the vector of the present invention is formulated in a delivery system vehicle. According to other embodiments, the nucleic acid construct of the present invention is formulated in a delivery system vehicle. According to yet farther embodiments, the set of anti-pathogenic staple nucleic acids is formulated in a delivery system vehicle. According to one embodiment, the delivery system is selected from liposomes, micelles, nanoparticle, viral nanoperticals, carbonano tubes, aptamer, polymer drug conjugates, dendrimers, gelatin capsules, proliposomes, self-assemblies, microspheres, gels, cyclodextrins, microspheres, nanostructures, virosomes, polymeric micelles, and chitosan.

According to any one of the above embodiments, the staple nucleic acids are obtained upon transcription of the nucleic acid construct. According to one embodiment, each staple nucleic acids is transcribed separately to form separate staple nucleic acids. According to some embodiments, the resulted RNA molecule transcribed to form a plurality of combined nucleic acid staples, e.g. 2, 3, 4, or 5 combined nucleic acid staples. According to some embodiments, the resulted RNA molecule is spliced or cleaved to obtain separate staples.

According to a further aspect, the present invention provides a method for treating a pathogen comprising contacting the pathogen with the set of staple nucleic acids, with nucleic acid construct or the vector of the present invention. According to some embodiments, contacting pathogen with the set of staple nucleic acids, construct or vector comprises transforming or infecting the pathogen with said set, nucleic acid construct or vector.

According to one embodiments, contacting a pathogen with the set of anti-pathogenic staples, with nucleic acid construct or the vector of the present invention comprises transforming or infecting the pathogen with said set, nucleic acid construct or vector.

The terms “transformation”, “transforming”, “transfection”, “transduction”, “transfecting” or “transducing” can be used interchangeably and are defined as a process of introducing or internalization of a nucleic acid molecule to a cell. Nucleic acids are introduced to a cell using non-viral or viral-based methods. The nucleic acid molecules may be sequences encoding to RNA. Non-viral methods of transformation include any appropriate transfection method that does not use viral DNA or viral particles as a delivery system to introduce the nucleic acid molecule, such as staple, construct or vector into the cell. Exemplary non-viral transfection methods include calcium phosphate transfection, liposomal transfection, nucleofection, sonoporation, transfection through heat shock, magnetifection, electroporation and cellular uptake. According to some embodiments, the nucleic acid molecules delivered/introduced into the cell using a transfection reagent. According to some embodiments, the transfection reagent is DOTAP, Alveofact, Lipofectamine, DharmaFECT, Cellfectine, nanoparticle, liposome, Polymeric particle, etc. According to some embodiments, transforming comprises internalizing of the nucleic acid using permeability enhancing molecules or vehicle. In some embodiments, the nucleic acid molecules are introduced into a cell using electroporation following standard procedures well known in the art. In some embodiments, the nucleic acid molecules are introduced into a cell using heat-shock following standard procedures well known in the art. In some embodiments, the nucleic acid molecules are introduced into a cell using permeability-enhancing moiety. In other embodiments, the nucleic acid molecules were selected to penetrate cells. For viral-based methods of transformation any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art.

According to one embodiment, the pathogen is selected from a bacteria, parasite and fungi.

The term “treating a pathogen” as used herein refers to killing the pathogen as well as inhibiting or preventing pathogen's growth, division, fission, replication and spread.

Thus in one embodiment, the present invention provides a method for killing a bacteria comprising contacting the bacteria with the set of antibacterial staple nucleic acids, with a nucleic acid construct or the vector of the present invention.

According to one embodiment, the term “treating bacteria” as used herein refers to killing bacteria as well as inhibiting or preventing bacteria growth, growth, division, fission, and replication. According to one embodiment, the present invention provides a method for treating bacteria comprising contacting the bacteria with a set of antibacterial nucleic acids, with the nucleic acid construct or with the vector of the present invention.

The term “treating a fungi” as used herein refers to killing the fungi as well as inhibiting or preventing fungi's growth, reproduction, replication and spread.

The term “treating a parasite” as used herein refers to killing the parasite as well as inhibiting or preventing parasite's growth, reproduction, replication and spread.

According to some embodiments, the bacteria are gram positive bacteria.

According to one embodiment, the gram positive bacteria are selected from Streptococcus, Staphylococcus, Enterococcus, Gram positive cocci, and Peptostreptococcus. Further optionally, the gram-positive bacteria is selected from beta-hemolytic Streptococcus, coagulase negative Staphylococcus, Enterococcus faecalis (VSE), Staphylococcus aureus, and Streptococcus pyogenes. Still further optionally, the gram-positive bacteria is selected from methicillin-sensitive Staphylococcus aureus (MSSA), and methicillin-resistant Staphylococcus aureus (MRSA), Staphylococcus aureus, Staphylococcus epidermis and other coagulase-negative staphylococci, Streptococcus pyogenes, Streptococcus pneumoniae, Streptococcus agalactiae, and Enterococcus.

According to other embodiments, the bacteria are gram negative bacteria. According to one embodiment, the gram negative bacteria are selected from the group consisting of: Actinobacillus, Aeromonas, Anaplasma, Arcobacter, Avibacterium, Bacteroides, Bartonella, Bordetella, Borrelia, Brachyspira, Brucella, Campylobacter, Capnocytophaga, Chlamydia, Chlamydophila, Chryseobacterium, Coxiella, Cytophaga, Dichelobacter, Edwardsiella, Ehrlichia, Escherichia, Flavobacterium, Francisella, Fusobacterium, Gallibacterium, Haemophilus, Histophilus, Klebsiella, Lawsonia, Leptospira, Mannheimia, Megasphaera, Moraxella, Neorickettsia, Nicoletella, Ornithobacterium, Pasteurella, Photobacterium, Piscichlamydia, Piscirickettsia, Porphyromonas, Prevotella, Proteus, Pseudomonas, Rickettsia, Riemerella, Salmonella, Streptobacillus, Tenacibaculum, Vibrio and Yersinia.

Further optionally, the gram-positive bacteria are selected from Staphylococcus spp, Streptococci, Enterococcus spp, Leuconostoc spp, Corynebacterium spp, Arcanobacteria spp, Trueperella spp, Rhodococcus spp, Bacillus spp, Anaerobic Cocci, Anaerobic Gram-Positive Nonsporulating Bacilli, Actinomyces spp, Clostridium spp, Nocardia spp, Erysipelothrix spp, Listeria spp, Kytococcus spp, Mycoplasma spp, Ureaplasma spp, and Mycobacterium spp.

According to some embodiments, the parasite is selected from protozoa, helminths or ectoparasites, e.g. toxoplasmosis, malaria, African trypanosomiasis, Chagas disease, leishmaniasis, schistosomiasis, amebiasis, giardiasis, clonorchiasis, opisthorchiasis, paragonimiasis, fasciolopsiasis, lymphatic filariasis, onchocerciasis, dracunculiasis, ascariasis, trichuriasis, stronglyoidiasis, trichostrongyliasis, trichomoniasis or cestodiasis.

According to other embodiments, the fungi is selected from the genera Trichophyton, Tinea, Microsporum, Epidermophyton, Aspergillus, Histoplasma, Cryptococcus, Microsporum, Candida, Malassezia, Trichosporon, Rhodotorula, Torulopsis, Blastomyces, Paracoccidioides, and Coccidioides, Trichophyton, Tinea, Microsporum, Epidermophyton; Cryptococcus, Candida, Paracoccidioides, Coccidioides, Trichophyton rubrum, Cryptococcus neoformans, Candida albicans, Paracoccidioides brasiliensis, and Coccidioides immitis.

According to some embodiments, contacting comprises incorporating the set of anti-pathogenic nucleic acids or nucleic acid construct or the vector into the pathogen. According to one embodiment, the pathogen is bacteria.

According to another aspect the present invention provides a composition comprising at least one of (i) at least one set of anti-pathogenic staple nucleic acids of the present invention; (ii) at least one nucleic acid construct comprising the sequences or reverse sequences of the set of anti-pathogenic staple nucleic acids; or (iii) at least one vector of the present invention. According to one embodiment, the composition comprises a plurality of sets of anti-pathogenic staple nucleic acids of the present invention. According to another embodiment, the composition comprises a plurality of nucleic acid constructs each comprising the set of anti-pathogenic staple nucleic acids of the present invention. According to a further embodiment, the composition comprises a plurality of vectors each comprising the set of anti-pathogenic staple nucleic acids of the present invention. According to one embodiment, the vector is a plasmid or a phage. According to one embodiment, the set of staples comprises a plurality of different staple nucleic acids that bind specifically pathogenic rRNA molecule(s) to form nucleic acid origami nanostructure(s). According to one embodiment, the set of staples comprises a plurality of different staple nucleic acids that bind specifically to one or more pathogenic mRNA molecules to form one or more nucleic acid origami nanostructure(s).

According to some embodiments, the set of staples comprises staples having SEQ ID NOs: 2-54. According to other embodiments, the set of staples comprises staples having SEQ ID NOs: 55-81. According to some embodiments, the set of anti-pathogenic staples comprises from 10 to 90% of staples having SEQ ID NOs: 2-54. According to other embodiments, the set of anti-pathogenic staples comprises from 10 to 90% of staples having SEQ ID NOs: 55-81. According to one embodiment, the set of staples comprises staples having SEQ ID NOs: 82-85. According to another embodiment, the set of staples comprises staples having SEQ ID NOs: 86-89. According to one embodiment, the set of staples comprises staples having SEQ ID NOs: 92-95. According to a further embodiment, the nucleic acid construct comprises nucleic acid sequence SEQ ID NO: 2-54. According to a further embodiment, the nucleic acid construct comprises nucleic acid sequences SEQ ID NO: 55-81. According to some embodiments, the set of anti-pathogenic staples construct comprises from 10 to 90% of staples having SEQ ID NOs: 2-54. According to other embodiments, the construct of anti-pathogenic staples comprises from 10 to 90% of staples having SEQ ID NOs: 55-81. According to a further embodiment, the nucleic acid construct comprises nucleic acid sequence SEQ ID NO: 91. According to some embodiments, the staples of the set of staples are RNA nucleic acid staples. In some embodiments, the set of staples are RNA nucleic acids corresponding to sequences selected from SEQ ID NOs: 2-55, SEQ ID NOs: 56-81, SEQ ID NOs: 82-85, SEQ ID NOs: 92-95, and SEQ ID NOs: 86-89, in which thymidine (T) is substituted with uridine (U). According to some embodiments, the set of staples comprises staple nucleic acids having sequences SEQ ID NO: 96-99. According to one embodiment, the set comprises staple nucleic acids having sequences SEQ ID NOs: 2-95. According to one embodiment, the set comprises staple nucleic acids, wherein said staples are RNA staples corresponding to sequences SEQ ID NOs: 2-95, in which T is substituted for U. According to another embodiment, the set comprises a combination of nucleic acids having sequences SEQ ID NOs: 2-95 and RNA staples having nucleic acids sequences corresponding to SEQ ID NOs: 2-95. According to one embodiment, the set of staples comprises from about 10% to about 99%, about 20% to about 95%, about 30% to about 90%, about 40% to about 85%, about 50% to about 80% or about 60% to about 70% of staples of said sets. According to one embodiment, the set of staples comprises from about 10% to about 99%, about 20% to about 95%, about 30% to about 90%, about 40% to about 85%, about 50% to about 80% or about 60% to about 70% of staples of a set of staple nucleic acids having the sequences SEQ ID NOs: 2-95. According to one embodiment, the set comprises staple nucleic acids having sequences SEQ ID NOs: 103-104 or SEQ ID NOs: 105-106 or SEQ ID NOs: 107-108 or SEQ ID NOs: 109-110 or any combination thereof.

According to any one of the above embodiments, the composition is a pharmaceutical composition further comprising a pharmaceutically acceptable carrier. According to another embodiment, the composition is an agricultural composition, further comprising an agriculturally-acceptable carrier.

According to another embodiment, the pharmaceutical composition comprising at least one of (i) at least one set of antibacterial nucleic acids of the present invention; (ii) at least one nucleic acid construct comprising the sequences or reverse sequences of the set of antibacterial nucleic acids, or (iii) at least one vector comprising the nucleic acid construct comprising the set of anti-bacterial nucleic acids.

The term “pharmaceutical composition” as used herein refers to a composition comprising at least one active ingredient such as a set of staples nucleic acids, nucleic acid construct or vector as disclosed herein optionally formulated together with one or more pharmaceutically acceptable carriers.

The term “agricultural composition” refers to a material or a combination of materials that are capable of improving the rate of growth or health of plants, increasing the yields of plants or their fruits, and/or improving or change the environments where the plants grow. In one embodiment, the agricultural composition can prevent, inhibit, or ameliorate a plant disease that affects the health, growth, and/or yield of a plant.

The term “agriculturally-acceptable carrier” covers all adjuvants, inert components, dispersants, surfactants, tackifiers, binders, etc. that are ordinarily used in pesticide formulation technology; these are well known to those skilled in pesticide formulation. The formulations may be mixed with one or more solid or liquid adjuvants and prepared by various means, e.g., by homogeneously mixing, blending and/or grinding the pesticidal composition with suitable adjuvants using conventional formulation techniques.

According to another embodiment, the pharmaceutical composition comprising at least one of (i) at least one set of anti-fungal nucleic acids of the present invention; or (ii) at least one nucleic acid construct comprising the sequences or reverse sequences of the set of anti-fungal nucleic acids, or (iii) at least one vector comprising the nucleic acid construct comprising the set of anti-fungal nucleic acids.

According to another embodiment, the pharmaceutical composition comprising at least one of (i) at least one set of anti-parasite nucleic acids of the present invention; or (ii) at least one nucleic acid construct comprising the sequences or reverse sequences of the set of anti-parasite nucleic acids, or (iii) at least one vector comprising the nucleic acid construct comprising the set of anti-parasite nucleic acids.

Formulation of the pharmaceutical composition may be adjusted according to applications. In particular, the pharmaceutical composition may be formulated using a method known in the art so as to provide induced, rapid, continuous or delayed release of the active ingredient after administration to mammals.

According to any one of the above embodiments, the pharmaceutical composition is in a form selected from the group consisting of tablets, pills, capsules, pellets, granules, powders, lozenges, sachets, cachets, elixirs, suspensions, dispersions, emulsions, solutions, infusions, syrups, aerosols, ophthalmic ointments, ointments, soft and hard gelatin capsules, suppositories, sterile injectable solutions, and sterile packaged powders.

According to any one of the above embodiments, the pharmaceutical composition is suitable for administration via a route selected from the group consisting of oral, rectal, intramuscular, subcutaneous, intravenous, inrtaperitoneal, intranasal, intraarterial, intravesicle, intraocular, transdermal and topical.

The composition for oral administration may be in a form of tablets, troches, lozenges, aqueous, or oily suspensions, dispersible powders or granules, emulsions, hard or soft capsules, or syrups or elixirs. Pharmaceutical compositions intended for oral use may be prepared according to any method known to the art for the manufacture of pharmaceutical compositions and may further comprise one or more agents selected from sweetening agents, flavoring agents, coloring agents and preserving agents in order to provide pharmaceutically elegant and palatable preparations. Tablets contain the active agent in admixture with non-toxic pharmaceutically acceptable excipients, which are suitable for the manufacture of tablets. These excipients may be, e.g., inert diluents such as calcium carbonate, sodium carbonate, lactose, calcium phosphate, or sodium phosphate; granulating and disintegrating agents, e.g., corn starch or alginic acid; binders; and lubricating agents. The tablets are preferably coated utilizing known techniques to delay disintegration and absorption in the gastrointestinal tract and thereby provide an extended release of the drug over a longer period.

The term “pharmaceutically acceptable carrier” or “pharmaceutically acceptable excipient” as used herein refers to any and all solvents, dispersion media, preservatives, antioxidants, coatings, isotonic and absorption delaying agents, surfactants, fillers, disintegrants, binders, diluents, lubricants, glidants, pH adjusting agents, buffering agents, enhancers, wetting agents, solubilizing agents, surfactants, antioxidants the like, that are compatible with pharmaceutical administration. The use of such media and agents for pharmaceutically active substances is well known in the art. The compositions may contain other active compounds providing supplemental, additional, or enhanced therapeutic functions. Solid carriers or excipients are, for example, lactose, starch or talcum or liquid carriers such as, for example, water, fatty oils or liquid paraffins.

Other carriers or excipients which may be used include, but are not limited to, materials derived from animal or vegetable proteins, such as the gelatins, dextrins and soy, wheat and psyllium seed proteins; gums such as acacia, guar, agar, and xanthan; polysaccharides; alginates; carboxymethylcelluloses; carrageenans; dextrans; pectins; synthetic polymers such as polyvinylpyrrolidone; polypeptide/protein or polysaccharide complexes such as gelatin-acacia complexes; sugars such as mannitol, dextrose, galactose and trehalose; cyclic sugars such as cyclodextrin; inorganic salts such as sodium phosphate, sodium chloride and aluminium silicates; and amino acids having from 2 to 12 carbon atoms and derivatives thereof such as, but not limited to, glycine, L-alanine, L-aspartic acid, L-glutamic acid, L-hydroxyproline, L-isoleucine, L-leucine and L-phenylalanine. Each possibility represents a separate embodiment of the present invention.

Solutions or suspensions used for parenteral, intradermal, or subcutaneous application typically include the following components: a sterile diluent such as water for injection, saline solution, fixed oils, polyethylene glycols, glycerine, propylene glycol (or other synthetic solvents), antibacterial agents (e.g., benzyl alcohol, methyl parabens), antioxidants (e.g., ascorbic acid, sodium bisulfite), chelating agents (e.g., ethylenediaminetetraacetic acid), buffers (e.g., acetates, citrates, phosphates), and agents that adjust tonicity (e.g., sodium chloride, dextrose). The pH can be adjusted with acids or bases, such as hydrochloric acid or sodium hydroxide, for example. The parenteral preparation can be enclosed in ampules, disposable syringes or multiple dose glass or plastic vials.

Pharmaceutical compositions adapted for parenteral administration include, but are not limited to, aqueous and non-aqueous sterile injectable solutions or suspensions, which can contain antioxidants, buffers, bacteriostats and solutes that render the compositions substantially isotonic with the blood of an intended recipient. Such compositions can also comprise water, alcohols, polyols, glycerin and vegetable oils, for example. Extemporaneous injection solutions and suspensions can be prepared from sterile powders, granules and tablets. Such compositions preferably comprise a therapeutically effective amount of a compound of the invention and/or other therapeutic agent(s), together with a suitable amount of carrier so as to provide the form for proper administration to the subject.

The terms “pharmaceutically acceptable” and “pharmacologically acceptable” include molecular entities and compositions that do not produce an adverse, allergic, or other untoward reactions when administered to an animal, or human, as appropriate. For human administration, preparations should meet sterility, pyrogenicity, general safety, and purity standards as required by a government drug regulatory agency, e.g., the United States Food and Drug Administration (FDA) Office of Biologics standards.

According to one embodiment, the present invention provides a pharmaceutical composition comprising at least one set of anti-pathogenic nucleic acids, wherein the set comprises a plurality of different staple nucleic acids, wherein said staple nucleic acids bind specifically to one or more pathogenic ribosomal RNA (rRNA) to form one or more nucleic acid origami nanostructure(s). According to one embodiment, the pathogenic rRNA is selected from 16S, 23S, 5S, 18S, 28S, 26S, 25S, 5.8S rRNA and pre-rRNA. According to another embodiment, the staple nucleic acids are selected from DNA and RNA nucleic acids. According to a further embodiment, the set comprises from 2 to 1000 or from 2 to 400 different staple nucleic acids. According to one embodiment, the bacterial rRNA is selected from 16S, 23S, 5S rRNA and pre-rRNA. According to another embodiment, the staple nucleic acids are selected from DNA and RNA nucleic acids. According to a further embodiment, the set comprises from 2 to 400 different staple nucleic acids. According to a certain embodiment, the set comprises staple nucleic acids having the sequences SEQ ID NO: 2 to 54 or SEQ ID NOs: 55-81. According to some embodiments, the set of anti-pathogenic staples comprise from 10 to 90% of staples having SEQ ID NOs: 2-54 or SEQ ID NOs: 55-81. According to one embodiment, the set of staples comprises staples having SEQ ID NOs: 82-85, SEQ ID NOs: 92-95 or SEQ ID NOs: 86-89. According to some embodiments, the staples of the set of staples are RNA nucleic acid staples. In some embodiments, the set of staples are RNA nucleic acids corresponding to sequences selected from SEQ ID NOs: 2-55, SEQ ID NOs: 56-81, SEQ ID NOs: 82-85, SEQ ID NOs: 92-95, and SEQ ID NOs: 86-89, in which thymidine (T) is substituted with uridine (U). According to some embodiments, the set of staples comprises staple nucleic acids having sequences SEQ ID NO: 96-99. According to one embodiment, the set comprises staple nucleic acids having sequences SEQ ID NOs: 2-95. According to one embodiment, the set comprises staple nucleic acids, wherein said staples are RNA staples corresponding to sequences SEQ ID NOs: 2-95, in which T is substituted for U. According to another embodiment, the set comprises a combination of nucleic acids having sequences SEQ ID NOs: 2-95 and RNA staples having nucleic acids sequences corresponding to SEQ ID NOs: 2-95. According to one embodiment, the set of staples comprises from about 10% to about 99%, about 20% to about 95%, about 30% to about 90%, about 40% to about 85%, about 50% to about 80% or about 60% to about 70% of staples of said sets. According to one embodiment, the set of staples comprises from about 10% to about 99%, about 20% to about 95%, about 30% to about 90%, about 40% to about 85%, about 50% to about 80% or about 60% to about 70% of staples of a set of staple nucleic acids having the sequences SEQ ID NOs: 2-95. According to one embodiment, the set comprises staple nucleic acids having sequences SEQ ID NOs: 103-104 or SEQ ID NOs: 105-106 or SEQ ID NOs: 107-108 or SEQ ID NOs: 109-110.

According to one embodiment, the present invention provides a pharmaceutical composition comprising at least one set of anti-fungal nucleic acids, wherein the set comprises a plurality of different staple nucleic acids, wherein said staple nucleic acids bind specifically to fungal ribosomal RNA (rRNA), messenger RNA molecules or pre-RNA to form one or more nucleic acid origami nanostructure(s). According to one embodiment, the present invention provides a pharmaceutical composition comprising at least one set of anti-parasitic nucleic acids, wherein the set comprises a plurality of different staple nucleic acids, wherein said staple nucleic acids bind specifically to parasite ribosomal RNA (rRNA) messenger RNA molecule(s) to form nucleic acid origami nanostructure(s). According to one embodiment, the pharmaceutical composition comprises a construct comprising such a set. According to another embodiment, the pharmaceutical composition comprises a vector comprising said nucleic acid construct. According to any one of the above embodiments, the origami nanostructure(s) are obtained in cellulo. According to any one of the above embodiments, the staples are introduced into the cell via permeability-enhancing moiety. According to yet another embodiments, the staples were selected to enter the cell.

According to one embodiment, the present invention provides a pharmaceutical composition comprising at least one nucleic acid construct comprising the sequences or reverse sequences of a set of anti-pathogenic staple nucleic acids, wherein said staple nucleic acids bind specifically to pathogenic RNA such as rRNA, pre-rRNA or mRNA molecule(s) to form nucleic acid origami nanostructure(s). According to another embodiment, the present invention provides a pharmaceutical composition comprising at least one nucleic acid construct comprising the sequences of a set of antibacterial staple nucleic acids that bind specifically to a bacterial ribosomal RNA (rRNA) molecule(s) to form nucleic acid origami nanostructure(s) in cellulo. According to a certain embodiment, the nucleic acid contract comprises a set of staples comprising nucleic acids having the sequences SEQ ID NO: 2 to 54 or SEQ ID NOs: 55-81. According to some embodiments, the set of anti-pathogenic staples comprise from 10 to 90% of staples having SEQ ID NOs: 2-54 or SEQ ID NOs: 55-81. According to one embodiment, the nucleic acid contract comprises a set of staples comprising staples having SEQ ID NOs: 82-85, SEQ ID NOs: 92-95 or SEQ ID NOs: 86-89. According some embodiments, the nucleic acid construct comprises nucleic acid sequence SEQ ID NO: 91. According to one embodiment, the set comprises staple nucleic acids having sequences SEQ ID NOs: 103-104 or SEQ ID NOs: 105-106 or SEQ ID NOs: 107-108 or SEQ ID NOs: 109-110.

According to one embodiment, the present invention provides a pharmaceutical composition comprising at least one nucleic acid construct comprising the reverse sequences of a set of antibacterial nucleic acids, wherein said staple nucleic acids bind specifically to bacterial ribosomal RNA (rRNA) molecule(s) to form nucleic acid origami nanostructure(s).

According to another embodiment, the present invention provides a pharmaceutical composition comprising at least one nucleic acid construct comprising the sequences of a set of antifungal or antiparasitic staple nucleic acids that bind specifically to fungal or parasitic mRNA molecule(s) to form nucleic acid origami nanostructure(s).

According to one embodiment, the present invention provides a pharmaceutical composition comprising at least one nucleic acid construct comprising the reverse sequences of a set of antifungal or antiparasitic staple nucleic acids that bind specifically to a fungal or parasitic ribosomal RNA (rRNA) to form a nucleic acid origami nanostructure.

According to a further embodiment, the present invention provides a pharmaceutical composition comprising the at least one vector comprising the nucleic acid construct comprising the sequences or the reverse sequences of a set of anti-pathogenic nucleic acids, wherein said staple nucleic acids bind specifically to pathogenic ribosomal RNA (rRNA) molecule(s) to form nucleic acid origami nanostructure(s). According to one embodiment, the pathogenic rRNA is bacterial, fungal or parasitic rRNA. According to one embodiment, the vector is a plasmid. According to another embodiment, the vector is a phage. According to a further embodiment, the vector is a shuttle vector. The term “shuttle vector” is a vector (usually a plasmid) constructed so that it can propagate in two different host species.

According to a further embodiment, the present invention provides a pharmaceutical composition comprising the at least one vector comprising the nucleic acid construct comprising the sequences or the reverse sequences of a set of antibacterial nucleic acids, wherein said staple nucleic acids bind specifically to a bacterial ribosomal RNA (rRNA) to form a nucleic acid origami nanostructure. According to one embodiment, the vector is a plasmid. According to another embodiment, the vector is a phage. According to any one of the above embodiments, the pharmaceutical composition is formulated to enhance the permeability of the vector, e.g. plasmid, into bacterial cell.

According to a further embodiment, the present invention provides a pharmaceutical composition comprising a set of anti-pathogenic staple nucleic acids, wherein the set comprises a plurality of different staple nucleic acids that bind specifically to one or more pathogenic mRNA, or pre-rRNA molecules to form a nucleic acid origami nanostructure. According to one embodiment, the pharmaceutical composition comprises a construct comprising such a set. According to another embodiment, the pharmaceutical composition comprises a vector comprising said nucleic acid construct.

According to any one of the above embodiments, the pharmaceutical composition is for use in treating a pathogenic infection. According to one embodiment, the pathogenic infection is a bacterial infection, fungal infection or parasitic infection. According to some embodiments, the pharmaceutical composition may be administered by any known method, as described herein.

According to any one of the above embodiments, the pharmaceutical composition is for use in treating a bacterial infection. Thus, in one embodiment, the present invention provides a pharmaceutical composition comprising a plurality of sets of anti-pathogenic staple nucleic acids, for use in treating a bacterial infection, wherein the set comprises a plurality of different staple nucleic acids that bind specifically pathogenic rRNA molecules to form a nucleic acid origami nanostructure. According to one embodiment, the set of staples comprises staple nucleic acids having nucleic acid sequences SEQ ID NOs: 2-54. According to another embodiment, the set of staples comprises staple nucleic acids having nucleic acid sequences SEQ ID NOs: 55-81. According to a further embodiment, the set of staples comprises staple nucleic acids having nucleic acid sequences SEQ ID NOs: 82-85. According to yet another embodiment, the set of staples comprises staple nucleic acids having nucleic acid sequences SEQ ID NOs: 86-89. According to a further embodiment, the set of staples comprises staple nucleic acids having nucleic acid sequences and SEQ ID NOs: 92-95. According to some embodiments, the set comprises from 10% to 90%, from 30% to 80% or from 40% to 60% of staples of the above sets. According to one embodiment, the set comprises a staple nucleic acids having sequences SEQ ID NOs: 2-95. According to one embodiment, the set of staples comprises from about 10% to about 99%, about 20% to about 95%, about 30% to about 90%, about 40% to about 85%, about 50% to about 80% or about 60% to about 70% of staples of a set of staples nucleic acids having the sequences SEQ ID NOs: 2-95. According to one embodiment, the set comprises any combination of sets of staples selected from a set comprising sequences SEQ ID NOs: 2-55, SEQ ID NOs: 56-81, SEQ ID NOs: 82-85, SEQ ID NOs: 92-95, and SEQ ID NOs: 86-89. According to some embodiments, the staples of the set of staples are RNA nucleic acid staples. In some embodiments, the set of staples are RNA nucleic acids corresponding to sequences selected from SEQ ID NOs: 2-55, SEQ ID NOs: 56-81, SEQ ID NOs: 82-85, SEQ ID NOs: 92-95, and SEQ ID NOs: 86-89, in which thymidine (T) is substituted with uridine (U). According to some embodiments, the set of staples comprises staple nucleic acids having sequences SEQ ID NO: 96-99. According to one embodiment, the set comprises staple nucleic acids having sequences SEQ ID NOs: 2-95. According to one embodiment, the set comprises staple nucleic acids, wherein said staples are RNA staples corresponding to sequences SEQ ID NOs: 2-95, in which T is substituted for U. According to another embodiment, the set comprises a combination of nucleic acids having sequences SEQ ID NOs: 2-95 and RNA staples having nucleic acids sequences corresponding to SEQ ID NOs: 2-95. According to one embodiment, the set of staples comprises from about 10% to about 99%, about 20% to about 95%, about 30% to about 90%, about 40% to about 85%, about 50% to about 80% or about 60% to about 70% of staples of said sets. According to one embodiment, the set of staples comprises from about 10% to about 99%, about 20% to about 95%, about 30% to about 90%, about 40% to about 85%, about 50% to about 80% or about 60% to about 70% of staples of a set of staple nucleic acids having the sequences SEQ ID NOs: 2-95. According to one embodiment, the set comprises staple nucleic acids having sequences SEQ ID NOs: 103-104 or SEQ ID NOs: 105-106 or SEQ ID NOs: 107-108 or SEQ ID NOs: 109-110.

According to some embodiments, the pharmaceutical composition is for use in treating a bacterial infection caused by gram positive or gram negative bacteria. According to some embodiments, the gram positive bacteria are selected from Streptococcus, Staphylococcus, Enterococcus, gram positive cocci, and Peptostreptococcus. According to other embodiments, the gram negative bacteria are selected Actinobacillus, Aeromonas, Anaplasma, Arcobacter, Avibacterium, Bacteroides, Bartonella, Bordetella, Borrelia, Brachyspira, Brucella, Campylobacter, Capnocytophaga, Chlamydia, Chlamydophila, Chryseobacterium, Coxiella, Cytophaga, Dichelobacter, Edwardsiella, Ehrlichia, Escherichia, Flavobacterium, Francisella, Fusobacterium, Gallibacterium, Haemophilus, Histophilus, Klebsiella, Lawsonia, Leptospira, Mannheimia, Megasphaera, Moraxella, Neorickettsia, Nicoletella, Ornithobacterium, Pasteurella, Photobacterium, Piscichlamydia, Piscirickettsia, Porphyromonas, Prevotella, Proteus, Pseudomonas, Rickettsia, Riemerella, Salmonella, Streptobacillus, Tenacibaculum, Vibrio and Yersinia.

According to some embodiments, the parasitic infection is an infection caused by protozoa, helminths or ectoparasites, e.g. toxoplasmosis, malaria, African trypanosomiasis, Chagas disease, leishmaniasis, schistosomiasis, amebiasis, giardiasis, clonorchiasis, opisthorchiasis, paragonimiasis, fasciolopsiasis, lymphatic filariasis, onchocerciasis, dracunculiasis, ascariasis, trichuriasis, stronglyoidiasis, trichostrongyliasis, trichomoniasis or cestodiasis.

According to other embodiments, the fungal infection is an infection caused a fungus selected from the genera Trichophyton, Tinea, Microsporum, Epidermophyton, Aspergillus, Histoplasma, Cryptococcus, Microsporum, Candida, Malassezia, Trichosporon, Rhodotorula, Torulopsis, Blastomyces, Paracoccidioides, and Coccidioides, Trichophyton, Tinea, Microsporum, Epidermophyton; Cryptococcus, Candida, Paracoccidioides, and Coccidioides. In certain embodiments, the subject the infection caused by a fungus selected from Trichophyton rubrum, Cryptococcus neoformans, Candida albicans, Paracoccidioides brasiliensis, and Coccidioides immitis.

According to another aspect, the present invention provides a method of treating a pathogenic infection in a subject in a need thereof comprising administering to said subject a therapeutically effective amount of sets of the present invention, of nucleic acid constructs of the present invention, or of vectors of the present invention, or the pharmaceutical composition of the present invention. According to some embodiments, the pharmaceutical composition comprises at least one of (i) at least one set of anti-pathogenic staple nucleic acids of the present invention; (ii) at least one nucleic acid construct comprising the sequences or reverse sequences of the set of anti-pathogenic staple nucleic acids; or (iii) at least one vector of the present invention; and a pharmaceutically acceptable excipient. According to some embodiments, the pathogen is bacteria. According to another embodiment, the pharmaceutical composition comprising at least one of (i) at least one set of antibacterial nucleic acids of the present invention; or (ii) at least one nucleic acid construct comprising; or (iii) at least one vector comprising the sequences or reverse sequences of the set of antibacterial nucleic acids set of the present invention. According to other embodiments, the pathogen is a fungi or a parasite.

The term “therapeutically effective amount” of a drug or agent is an amount of a drug or an agent that, when administered to a subject will have the intended therapeutic effect, e.g. anti-pathogenic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, the nature and extent of the cognitive impairment, and the therapeutics or combination of therapeutics selected for administration, and the mode of administration. The skilled worker can readily determine the effective amount for a given situation by routine experimentation.

According to one embodiment, the present invention provides a method of treating a pathogenic infection in a subject in a need thereof comprising administering to said subject a plurality of sets, wherein the set comprises staple nucleic acid molecules selected from SEQ ID NOs: 2-54, SEQ ID NOs: 55-81, SEQ ID NOs: 82-85, SEQ ID NOs: 86-89 and SEQ ID NOs: 92-95 or from 10% to 90%, from 30 to 80% or from 40 to 60% of staples of said set. According to one embodiment, the set comprises staple nucleic acids having sequences SEQ ID NOs: 103-104 or SEQ ID NOs: 105-106 or SEQ ID NOs: 107-108 or SEQ ID NOs: 109-110. According to one embodiment, the method comprises administering a pharmaceutical composition comprising said sets.

According to some embodiments, the method comprises administering the pharmaceutical composition via a route selected from the group consisting of oral, rectal, intramuscular, subcutaneous, intravenous, inrtaperitoneal, intranasal, intraarterial, intravesicle, intraocular, transdermal and topical.

According to another aspect, the present invention provides use of a set of anti-pathogenic staple nucleic acids for preparation of a medicament for use in treating a pathogenic infection, wherein the set comprises a plurality of different staple nucleic acids that bind specifically to one or more pathogenic RNA molecules to form a nucleic acid origami nanostructure, wherein the RNA is selected from ribosomal RNA (rRNA), pre-RNA, and mRNA. According to one embodiment, the set comprises staple nucleic acid molecules selected from SEQ ID NOs: 2-54, SEQ ID NOs: 55-81, SEQ ID NOs: 82-85, SEQ ID NOs: 86-89 and SEQ ID NOs: 92-95 or from 10% to 90%, from 30 to 80% or from 40 to 60% of staples of said set. According to another embodiment, the construct comprises SEQ ID NO:91. According to another embodiment, the pathogenic infection is a bacterial infection.

According to one embodiment, the set comprises a staple nucleic acids having sequences SEQ ID NOs: 2-95. According to one embodiment, the set of staples comprises from about 10% to about 99%, about 20% to about 95%, about 30% to about 90%, about 40% to about 85%, about 50% to about 80% or about 60% to about 70% of staples of a set of staples nucleic acids having the sequences SEQ ID NOs: 2-85 and 92-95. According to one embodiment, the set comprises any combination of sets of staples selected from a set comprising sequences SEQ ID NOs: 2-55, SEQ ID NOs: 56-81, SEQ ID NOs: 82-85, SEQ ID NOs: 92-95, and SEQ ID NOs: 86-89.

According to one aspect, the present invention provides a method of selecting membrane-permeable staples, the method comprises:

a) incubating E. coli with staple library at 37° C.;
b) centrifuging and washing the cells, optionally in PBS buffer, optionally 2 or more times, and further dispersing the cells in a small volume of the buffer;
c) adding buffer-DNase and of DNaseI, and incubating according to manufacturer instructions;
d) centrifuging the cells and removing the supernatant;
e) re-suspending the E. coli in sequencing buffer;
f) lysing cells;
g) cleaning and extracting the DNA from the mixture;
h) detecting the staples internalized into E. coli;
i) sequencing the staples.

According to some embodiments, E. coli are incubated with a library (set) of staples from 1 to 12 hours. According to one embodiment, E. coli are incubated with a library (set) of staples from 1 to 3 hours. According to another embodiment, the lysis is performed by heating at 85 to 100° C. According to some embodiments, the staples are marked. According to some embodiments, the staples comprises a primer for detection by PCR. According to some embodiments, the detection comprises preparation of DNA by PCR for sequencing. According to some embodiments, further to PCR, the primers are removed. According to another embodiment, the staples comprise a radioactive or fluorescent marker. According to some embodiments, the staples are capable of forming nucleic acid origami nanostructure(s) with rRNA. According to other embodiments, the staples are staples capable of forming nucleic acid origami nanostructure(s) with mRNA.

According to some embodiments, the procedure is repeated from 2 to 15 times.

The terms “comprising”, “comprise(s)”, “include(s)”, “having”, “has” and “contain(s),” are used herein interchangeably and have the meaning of “consisting at least in part of”. When interpreting each statement in this specification that includes the term “comprising”, features other than that or those prefaced by the term may also be present. Related terms such as “comprise” and “comprises” are to be interpreted in the same manner. The terms “have”, “has”, having” and “comprising” may also encompass the meaning of “consisting of” and “consisting essentially of”, and may be substituted by these terms. The term “consisting of” excludes any component, step or procedure not specifically delineated or listed. As used herein, the term “about” means within ±10% of the value that follows it. In some embodiments, term about “about” means within ±5% or about ±1% of the value that follows it.

This invention is further illustrated by the following examples, which should not be construed as limiting. Those skilled in the art will recognize, or be able to ascertain, using no more than routine experimentation, numerous equivalents to the specific substances and procedures described herein. Such equivalents are intended to be encompassed in the scope of the claims that follow the examples below.

### Examples

### Example 1. Folding Bacterial 16S rRNA Using Two Different Sets of DNA Staples

In this experiment the folding of rRNA-DNA structures according to folding protocol 1, using total RNA from E. coli DH5α comprising 16S rRNA was tested. The 16S rRNA (SEQ ID NO: 1) was used as a scaffold implementing 2 different set of staples: Set 1 (SEQ ID NOs: 2-54) and Set 2 (SEQ ID NOs 55-81) for folding. The folding was performed at 12.5 mM MgCl₂, using scaffold:set of staples ratio of 1:10 (20 nM:200 nM).

Protocol 1:
incubating at 60° C. for 1 min
incubating at 55° C. for 5 min
incubating at 50° C. for 10 min
incubating at 37° C. for 10 min; and
incubating at 25° C. for 10 min.

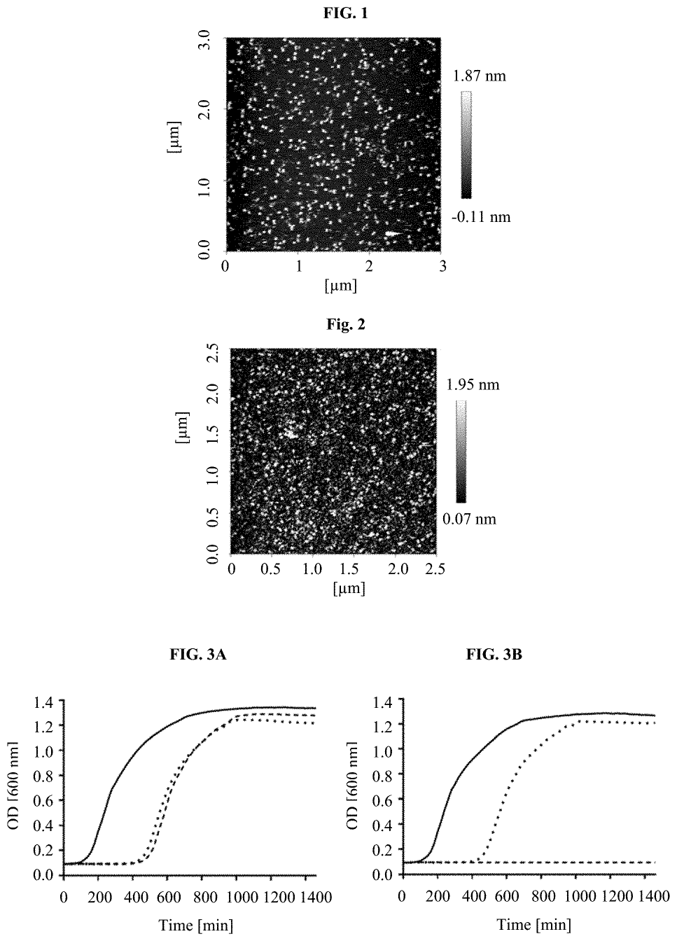

Following the folding, the resulted structures were incubated at 37° C. for 1-6 days. As can be seen from FIGS. 1 and 2 showing the resulted origami structures obtained after incubation for 5.5 days at 37° C., both sets of staples were effective in folding bacterial 16S rRNA.

### Example 2. Antibacterial Effect of Staples Nucleic Acids

A vector comprising 4 different staple sequences was prepared (sequences SEQ ID NO:82-85). Each staple was operable linked to a T7 promoter, an operator, and terminator defining a staple unit (sequences SEQ ID NO:86-89). The staple units were separated by a spacer. A contract comprising 4 different staple units was prepared (SEQ ID NO: 91). The construct was inserted into PJ281 vector (denoted as V1).

E. coli (BL21 (DE3) were transfected by heat shock with a vector backbone (PJ281 vector with a spacer—negative control, SEQ ID NO: 90) or with vector V1. Following transfection, BL21 (DE3) were grown overnight on 96 well plate in LB at 37° C. while shaking. 50 μg/mL kanamycin were added to each well 1 hour after initiation of the growth. Following 1 hour from addition of kanamycin, 1 mM IPTG were added to induce the transcription of the insert, and therefore the staples. FIG. 3 shows the results of growing of untransformed (continues curve) E. coli and E. coli transformed with V.Backbone (dot curve) or with V1 vector (dash curve). FIG. 3 shows growth of E. coli with 0 mM (un-induced—FIG. 3A) or 1 mM (induced—FIG. 3B) IPTG.

It can be clearly seen that upon induction and subsequently transcription of the staples, the growth of the transduced E. coli cells was completely inhibited.

### Example 3. Antibacterial Effect of Staples Nucleic Acids

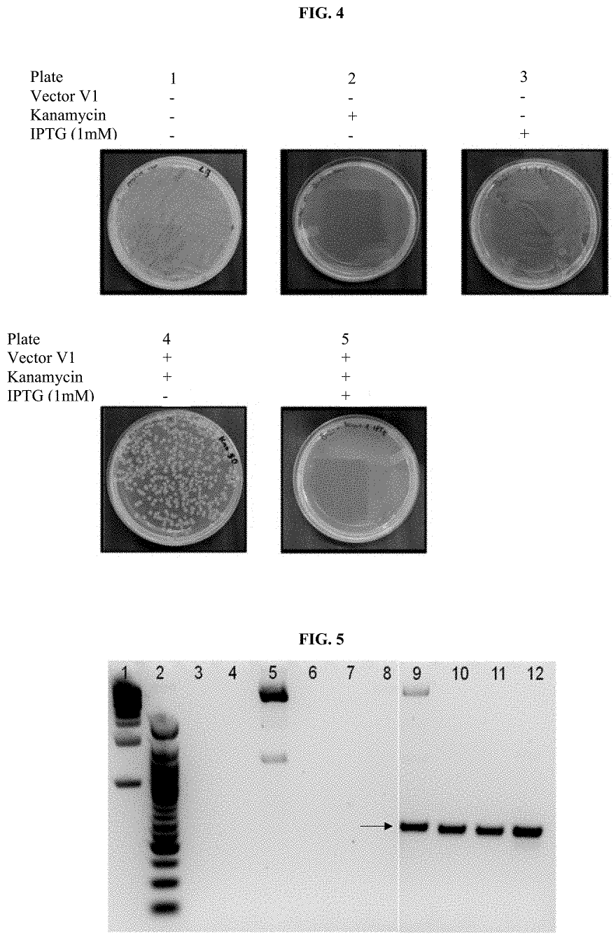

In a parallel experiment, transfected E. coli cells were grown on kanamycin LB agar plates (50 μg/mL kanamycin). The results are shown in FIG. 4. Plates 1 and plate 2 are positive and negative controls for growth of BL21 (DE3) without and with kanamycin 50 μg/mL respectively. Plate 3 demonstrate the growth of BL21 (DE3) on LB plate (without kanamycin) with 1 mM IPTG. As can be see IPTG did not affect the native BL21 (DE3) growth. Plates 4 and 5 compare the effect of addition of 1 mM IPTG on the growth of BL21 (DE3) transformed with V1 (comprising staples SEQ ID NO:86-89). As can be seen, E. coli comprising V1 (having kanamycin resistance) were perfectly viable. On the contrary, cells comprising V1 vectors that were induced and transcribed did not grow at all.

### Example 4. Validation of the Presence of Staples in Transduced E. coli

To validate the presence of 4 staples, described in Example 2 and 3, PCR for each staple in the total RNA extraction was performed using a specific set of primers for each staple.

Total RNA were extracted from BL21 (DE3) grown in LB with 50 μg/mL kanamycin and 1 mM IPTG to induce insert transcription. PCR were preformed to verify the presence of each staple in the total RNA extraction using a specific set of primers. FIG. 5 shows the results. Lane 1 and 2 of FIG. 5 represent 1 kb and 50 bp ladders respectively. Lane 3 and 4 are controls: lane 3 NTC for cDNA reaction to verify that there is no contamination in the cDNA kit. All the components besides the template (total RNA) were inserted to the reaction. Lane 4, is NTC for PCR to verify absence of contamination during PCR amplification. All the components besides the template (cDNA of the total RNA) were inserted to the reaction. Lanes 5-8 check cross-reactivity of each pair of primers (pair is configured as forward and reverse primers that amplify one of the four staples). Lanes 9-12 check the transcription of the 4 staples v1.st, v1.st2, v1.st3 and v1.st4 (SEQ ID NO:86-89) in BL21 (DE3) transformed with V1 respectively. The arrow indicates the staples bands.

### Example 5. Selection of Membrane-Permeable Staples

The membrane-permeable staples are selected as described below.

1. E. coli are grown over night in LB in growing tubes following which are divided in aliquots of 10⁹ cells/mL in LB to final volume of 1 mL (OD 1).
2. The E. coli are incubated with staple library for 1 hour in incubator at 37° C. while shaking. Each staple comprises a sequence complementary to a primer, used to identify the staple.
3. The cells are centrifuged at 13×g for 5-10 min, and washed with PBS and resuspended in 1 ml PBS.
4. 50 μl of reaction buffer-DNase and 1 μl of DNaseI are added according to manufacturer's instructions (incubate for 10 minutes in incubator at 37° C.
5. the cells are centrifuged at 13×g for 5-10 min, and the supernatant is removed.
6. The E. coli are re-suspend in sequencing buffer (usually DDW) to final volume of 50 μl.
7. The cells are lysed by heat at 95° C. for 10 minutes.
8. DNA is extracted and cleaned from the mixture (dilution in DDW) using DNA purification kit.
9. DNA is than prepared by PCR reaction for sequencing.
10. Primers are removed e.g. by extracting from or gel or PCR DNA purification kit.

### 12. Sequencing.

The selection procedure is repeated to obtain staples with higher permeability

### Example 6. Design of RNA Staples that Bind Staphylococcus aureus Ribosomal 5S

Design of RNA or DNA staples that bind to 5S ribosomal RNA of Staphylococcus aureus and form rRNA-RNA or rRNA-DNA origami structures was done using caDNAno software. The obtained staples were further conjugated with cholesterol TEG to improve their permeability into cells. The cholesterol TEG was connected to 3′. Alternatively, it may be connected to 5′ or to any other nucleotide, or to several nucleotides.

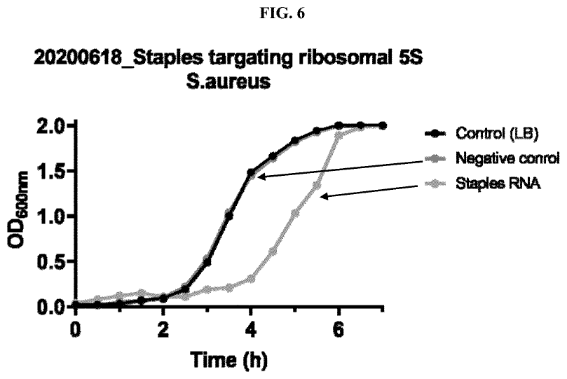

Efficacy of staples 1 and 2 on Staphylococcus aureus growth was tested as follows. Staphylococcus aureus was grown in tubes with 3 ml LB and incubated at 37° C. shaker with 250 RPM shaking overnight. On the following day the cells were diluted by 1:1000 in growing tubes with final volume of 2 ml(2 μl bacteria into 1998 μl LB). Next a 25 μl from a 100 μM stock staples, a control (LB) or negative control (random RNA oligos) were added, and the bacteria were grown at 37° C. at 250 RPM (final concentration of each staple was 1.25 μM). The OD was measured every 30 min. It can be clearly seen in FIG. 6 that the RNA staples inhibited the Staphylococcus aureus growth (red) as opposed to control sample (black) that represents the growth of Staphylococcus aureus in the absence of these staples.

This example clearly show that RNA staples may effectively inhibit the growth of bacteria. In addition it can be seen that RNA staples conjugated to cholesterol TEG were able penetrate into bacteria and inhibit to their growth.

Although the present invention has been described herein above by way of preferred embodiments thereof, it can be modified, without departing from the spirit and nature of the subject invention as defined in the appended claims.

1. A set of anti-pathogenic staple nucleic acids, wherein the set comprises a plurality of different staple nucleic acids that bind specifically to one or more pathogenic RNA molecules to form one or more nucleic acid origami nanostructures in cellulo, wherein the RNA is selected from ribosomal RNA (rRNA), pre-rRNA and mRNA.

2. The set according claim 1, wherein the staple nucleic acids are selected from DNA, RNA nucleic acids and any combination thereof.

3. The set according to claim 1, wherein the pathogen is bacteria.

4. The set according to claim 3, wherein the bacterial RNA is selected from 16S, 23S, 5S rRNA, mRNA and pre-rRNA.

5. The set according to claim 4, wherein the set comprises a set selected from (i) a set comprising staple nucleic acids having the sequences SEQ ID NOs: 2-54, (ii) a set comprising staple nucleic acids having the sequences SEQ ID NOs: 55-81, (iii) a set comprising staple nucleic acids having the sequences SEQ ID NOs: 82-85, (iv) a set comprising from 10% to 90% of staples of a set of staple nucleic acids having the sequences SEQ ID NOs: 2-54 or SEQ ID NOs: 55-81, (v) a set comprising from 10% to 90% of staples of a set of staple nucleic acids having the sequences SEQ ID NOs 2-81, (vi) a set comprising staple nucleic acids having the sequences SEQ ID NOs: 107-108, (vii) a set comprising staple nucleic acids having the sequences SEQ ID NOs: 109-110, and (vii) a combination thereof.

6. The set according to claim 1, wherein the staples of the set(s) bind either one RNA molecule or simultaneously to two or more different RNA molecules, optionally wherein each of said staple nucleic acid comprises at least 99%, 98%, 97%, 96%, 95%, 90%, 85%, 80%, 75%, 70%, 65%, 60%, 55%, or 50% sequence complementarity to each fragment of the scaffold nucleic acid to which it designed to bind.

7. The set according to claim 1, wherein the set forms one or more origami nanostructures.

8. The set according to claim 1, wherein the set is characterized by at least one of (i) the staples are selected from membrane-permeable staples, staples conjugated to a permeability-enhancing moiety, staples comprising modified nucleotides and any combination thereof, (ii) the set is formulated in a delivery system vehicle and (iii) both (i) and (ii).

9. The set according to claim 1, wherein at least one of the staples is operably linked to at least one of a promoter, operator or terminator.

10. The set according to claim 9, wherein the RNA is a bacterial rRNA and the set comprises staples having SEQ ID NOs: 86-89 or SEQ ID NOs: 92-95, optionally wherein each one of the staples is operably linked to a promoter and terminator.

11. The set according to claim 1, wherein the set comprises RNA staples corresponding to sequence of staples of a set selected from SEQ ID NOs: 2-54, SEQ ID NOs: 55-81, SEQ ID NO: 82-85, SEQ ID NO: 86-89, SEQ ID NOs: 92-95, and any combination thereof, in which T is substituted for U, or wherein the set comprises staple nucleic acids having the sequences SEQ ID NOs: 103-104 or SEQ ID NOs: 105-106, and any combination thereof.

12. A nucleic acid construct comprising the sequences or the reverse sequences of the staple nucleic acids of the set according claim 1, optionally comprising a spacer between each pair of staple sequences and/or wherein the construct is conjugated with a permeability-enhancing moiety.

13. The nucleic acid construct according to claim 12, comprising the nucleic acid sequence SEQ ID NO: 91 or RNA nucleic acid corresponding to SEQ ID NO: 91 in which T is substituted for U.

14. The nucleic acid construct according to claim 12, wherein the spacer forms a cleavable spacer or wherein the spacer has a nucleic acid sequence selected from SEQ ID NOs: 100, 101 and 102.

15. The nucleic acid construct according to claim 12, wherein the staple nucleic acids are obtained upon in cellulo transcription of the nucleic acid construct and optionally further splicing of the resulted RNA molecule or upon separate transcription of each staple nucleic acid.

16. A vector comprising the nucleic acid construct according to claim 12, optionally wherein the vector is conjugated with a permeability-enhancing moiety.

17. A method for treating a pathogen comprising contacting the pathogen with the set of anti-pathogenic staple nucleic acids according to claim 1 or a nucleic acid construct comprising thereof.

18. The method according to claim 17, wherein contacting comprises transforming, transfecting or infecting the pathogen.

19. A composition comprising the at least one set of anti-pathogenic nucleic acids according to claim 1, or a nucleic acid construct comprising thereof.

20. The composition according to claim 19, wherein the composition is a pharmaceutical composition.

21. A method of treating a pathogenic infection in a subject in a need thereof comprising administering to said subject a therapeutically effective amount of sets of staple nucleic acids according to claim 1 or the pharmaceutical composition comprising thereof.

22. The method of claim 21, wherein the pathogenic infection is a bacterial infection.
